(19) United States
(12) Patent Application Publication
(10) Pub. No.: US 2009/0247516 A1
(43) Pub. Date: Oct. 1, 2009

(54) 3- (BENZO [D] [1,3] DIOXOL-5-YLMETHYL) -4- (THIO) OXO-2- (THIO) OXO-AZOLIDIN-5-YLIDENE DERIVATIVES AS ANTIBACTERIAL AGENTS

(76) Inventors: Miha Kotnik, Ljubljana (SI); Marko Oblak, Ljubljana (SI); Petra Stefanic Anderluh, Ljubljana (SI); Andrej Prezelj, Ljubljana (SI); Jan Humljan, Metlika (SI); Ivan Plantan, Skofljica (SI); Uros Urleb, Ljubljana (SI); Tomaz Solmajer, Ljubljana (SI)

Correspondence Address: MANELLI DENISON & SELTER, 2000 M STREET NW SUITE 700, WASHINGTON, DC, 20036-3307, US

(21) Appl. No.: 12/442,970

(22) PCT Filed: Oct. 8, 2007
(86) PCT No.: PCT/EP07/60655

§ 371 Date: Mar. 26, 2009

(30) Foreign Application Priority Data
Oct. 10, 2006 (EP) 06122058.8

(51) Int. Cl. A61K 31/5377; C07D 471/04; A61K 31/519; C07D 417/14; A61K 31/4439; A61P 31/04

(52) U.S. Cl. 514/233.8; 544/282; 514/259.41; 546/269.7; 514/342; 544/133; 514/236.8; 544/131

(57) ABSTRACT

The present invention concerns novel azolidine compounds of Formula (I) wherein X denotes S, NH, CH2 or O, Y and W denotes each independently S or O, Q denotes CH or N, n and m are an integer from 0 to 2, and R1 and R2 denote independently from each other hydrogen, optionally substituted aryl, optionally substituted heterocyclyl, or an optionally substituted (C1-10) aliphatic group, R3 denotes hydrogen, lower alkyl, halogen, lower alkoxy, nitro, and R4 and R5 denote independently from each other hydrogen, halogen or alkyl, with the proviso that if one of R1 and R2 is hydrogen then the respective other one of R1 and R2 does not denote substituted or unsubstituted 2-furyland their salts, that are useful as pharmaceutical agents, e.g. as antibacterial agents, and to pharmaceutical compositions comprising such compounds.

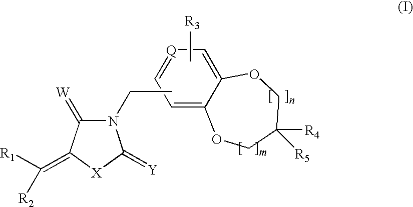

The present invention concerns novel azolidine compounds that are useful as pharmaceutical agents, e.g. as antibacterial agents, and to pharmaceutical compositions comprising such compounds.

The widespread emergence of pathogenic bacterial strains with resistance to antibiotics is becoming a serious threat for public health. As a result of resistance, some bacterial infections are either difficult to treat with existing antibiotics or even untreatable. Recent development of multi drug resistance in certain strains of bacteria, such as methicillin resistant Staphylococcus aureus (MRSA), methicillin resistant coagulase negative Staphylococci (MRCoNS), penicillin resistant Streptococcus pneumoniae, Mycobacterium tuberculosis, Pseudomonas aeruginosa, and multiply resistant Enterococcus faecium has risen the seriousness of this problem on a higher level. The appearance of vancomycin resistant Enterococcus (VRE) was particularly alarming because vancomycin was formerly the only effective antibiotic for treating this infection. Another resistant hospital-acquired pathogen is carbapenem-resistant Acinetobacter baumannii. (A. El Zoeiby, et al., Molecular Microbiology, 2003, 47, 1-12; F. M. Walsh, et al., Current Opinion in Microbiology, 2004, 7, 439-444.)

This situation has generated an urgent need to develop novel broad spectrum antibacterial agents preferably directed towards previously unexplored targets.

Since the essential bacterial cell-wall polymer peptidoglycan is exclusively found in prokaryotic cells the enzymes involved in the peptidoglycan sythesis, such as the Murein ligases MurD, MurC, MurE and MurF are excellent selective targets for an effective antibiotic against prokaryotic organisms.

The present invention relates to novel azolidine compounds being suitable as pharmaceuticals, e.g. as antibacterial agents.

Particularly, the present invention relates to a compound of formula

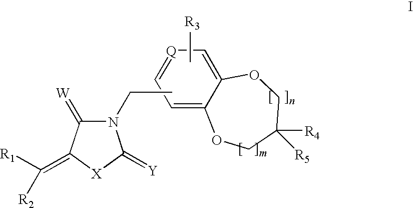

wherein
X denotes S, NH, CH₂ or O, preferably S or O
Y denotes S or O, preferably S
W denotes S or O, preferably O
Q denotes CH or N, preferably CH
n is an integer from 0 to 2,
m is an integer from 0 to 2, whereby the sum of n+m is not greater than 2, e.g. n is 1 and m is 1, or preferably n is 0 and m is 0,
R1 and R2 denote independently from each other hydrogen, optionally substituted aryl, optionally substituted heterocyclyl, or an optionally substituted (C₁₋₁₀) aliphatic group,
R3 denotes hydrogen, lower alkyl, halogen, lower alkoxy, nitro, and

R4 and R5 denote independently from each other hydrogen, halogen or alkyl, with the proviso that if one of R1 and R2 is hydrogen then the respective other one of R1 and R2 does not denote substituted or unsubstituted 2-furyl,

and pharmaceutically acceptable salts of a compound of formula I.

As used herein the following definitions shall apply unless otherwise specifically indicated. The term aliphatic means straight chained, branched or cyclic hydrocarbons which are completely saturated or which contain one or more units of unsaturation. For example, suitable aliphatic groups include substituted or unsubstituted linear, branched or cyclic alkyl, alkenyl, alkinyl groups and hybrids thereof such as (cycloalkyl)alkyl, (cycloalkenyl)alkyl or (cycloalkyl)alkenyl. Alkyl includes (C₁₋₂₀)alkyl, particularly (C₁₋₈)alkyl, such as lower alkyl. Lower alkyl includes (C₁₋₄)alkyl, such as methyl or ethyl. Alkenyl includes (C₂₋₂₀)alkenyl, particularly (C₂₋₈)alkenyl. Alkinyl includes (C₂₋₂₀)alkinyl, particularly (C₂₋₈)alkinyl. Cycloalkyl includes (C₃₋₁₈)cycloalkyl, particularly (C₃₋₁₀)cycloalkyl, such as (C₃₋₈)cycloalkyl, for example cyclohexyl, cyclopentyl or cyclopropyl.

Alkoxy includes (C₁₋₂₀)alkoxy such as (C₁₋₈)alkoxy, e.g. lower alkoxy. Lower alkoxy includes (C₁₋₄)alkoxy, in particular methoxy and ethoxy. Halogen includes fluoro, chloro, iodo and bromo such as fluoro and chloro. Alkyl, alkenyl, alkinyl and cycloalkyl include unsubstituted alkyl, alkenyl, alkinyl and cycloalkyl; and substituted alkyl, alkenyl and cycloalkyl, for example, substituted by halogen, hydroxy, alkoxy, amino, nitro, alkylamino, or dialkylamino. Halogenalkyl includes alkyl wherein at least one hydrogen atom and at maximum all halogen atoms are replaced by halogen, e.g. trihalomethyl such as trifluoromethyl.

The term heteroatom includes N, O and S.

Aryl includes (C₆₋₁₈)aryl, such as (C₆₋₁₀)aryl. In particular aryl includes phenyl and naphtyl, e.g. phenyl. Aryl may be unsubstituted aryl or substituted aryl, for example by alkoxy, aryloxy, alkylthio, hydroxy, alkyl, amino, alkylamino, dialkylamino, alkylsulfonamido, carboxy, halogen, haloalkyl, cycloalkyl, nitro, cyano, or heterocyclyl. Alkylamino and dialkylamino include alkylamino and dialkylamino wherein the alkyl parts are as defined above.

If not expressly otherwise indicated herein heterocyclyl includes aromatic or alicyclic heterocyclyl being monocyclic and having 3 to 7 members, preferably 5 to 6 members, containing at least one carbon atom and 1 to 4 heteroatoms beside the carbon atoms, or being bicyclic and having 8 to 10 members containing at least 3 carbon atoms and 1 to 7 heteroatoms. Heterocyclyl includes further unsubstituted heterocyclyl and substituted heterocyclyl, for example by oxo, alkoxy, aryloxy, alkylthio, hydroxy, thioxo, alkyl, amino, alkylamino, dialkylamino, alkylsulfonamido, carboxy, halogen, haloalkyl, cycloalkyl, nitro, cyano, isocyano or heterocyclyl. Examples of aromatic heterocyclyl include furanyl; thienyl; pyrrolyl; 2H-pyrrolyl; oxazolyl; thiazolyl; imidazolyl; pyrazolyl; isoxazolyl; isothiazolyl; 1,2,3-oxadiazolyl; 1,2,4-oxadiazolyl; 1,2,4-thiadiazolyl; 1,3,4-oxadiazolyl; 1,3,4-thiadiazolyl; 2H-pyranyl; 4H-pyranyl; 1,4-dioxan; 1,4-dithiane; pyridazinyl; pyrimidinyl, pyrazinyl, 1,2,3-triazinyl, pyridinyl, tetrazolyl. Examples of non-aromatic heterocyclyl include tetrahydrofuranyl, tetrahydrothienyl, morpholinyl, thiomorpholinyl, 2H-pyrrolinyl, 3H-pyrrolinyl, pyrrolidinyl, piperazinyl, piperidinyl, 2-pyrazolinyl, pyrazolidinyl, 3,6-dihydro-2H-pyranyl, 1,2,3,6-tetrahydro-pyridyl, thiazolidinyl, imidazolidinyl, 2-imidazolinyl, diazolone, N-substituted diazolone, phthalimidinyl, benzoxane, benzotriazolyl, benzopyrrolidinyl, benzopiperidinyl, 1,3-dihydroisobenzofuranyl, 1,3-dihydroisobenzothienyl, tetrahydroisoquinolinyl, decahydroisoquinolinyl or benzothianyl.

In a preferred embodiment one of the residues of the group consisting of R1 and R2 denotes hydrogen and the other one is defined as above, e.g. R1 denotes hydrogen and R2 is as defined above, or R2 denotes hydrogen and R1 is as defined above. Preferably R1 or R2 denote aryl or heterocyclyl. Aryl in this connection may be unsubstituted aryl such as phenyl, or substituted aryl, substituted one or several fold, e.g. 1 to 4 fold by hydroxy, alkoxy, alkylthio, aryloxy, alkyl, amino, alkylamino, dialkylamino, nitro, cyano, halogen, carboxy, alkylsulfonamido, heterocyclyl. If R1 or R2 preferably denote heterocyclyl then heterocyclyl may e.g. be pyridinyl, pyrimidinyl, oxazolyl, 1H-indol-3-yl, 1H-benzoimidazol-2-yl, imidazolyl, 1,2,4-triazolyl, benzo[d][1,3]dioxol-5-yl, 1,2,3-thiadiazolyl, thiazolyl, 4H-benzo[d][1.3]dioxinyl or pyrido[1.2a]pyrimidinyl, which may be unsubstituted or substituted one or several fold, e.g. 1 to 4 fold, by lower alkyl, oxo, cyano, nitro, halogen, morpholino, piperazino, hydroxy, alkoxy, aryloxy, amino, alkylamino, dialkylamino, (hydroxyalkyl)amino, (hydroxyalkoxyalkyl)amino or (heterocyclylalkyl)amino.

In a particularly preferred embodiment R1 is hydrogen and R2 is a group of formula

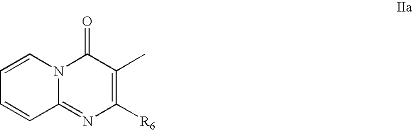

wherein R6 denotes amino, alkylamino, dialkylamino, (hydroxyalkyl)amino, di(hydroxyalkyl)amino, (heterocyclylalkyl)amino, (hydroxyalkoxyalkyl)amino or di(hydroxyalkoxyalkyl)amino. R6 may preferably be methylamino, ((tetrahydrofuran-2-yl)-methyl)amino, (hydroxy)(lower alkyl))amino or (2-hydroxyethoxy)ethyl)amino.

In another preferred embodiment one residue of the group consisting of R1 and R2 is hydrogen and the other one is aryl or a monocyclic or bicyclic aromatic heterocyclyl with the proviso that R1 or R2 is not 2-furanyl. E.g. aryl or a monocyclic or bicyclic aromatic heterocyclyl for R1 or R2 includes phenyl, pyridinyl, piperonyl, pyrimidinyl, triazinyl, pyrazolyl, imidazolyl, benzimidazolyl, thiazolyl, isothiazolyl, thiadiazolyl, benzodioxinyl, oxazolyl, isoxazolyl or oxadiazolyl, such as phenyl, thiadiazolyl, piperonyl, triazolyl, imidazolyl, benzodioxinyl, oxazolyl or pyridinyl. The aryl or monocyclic or bicyclic aromatic heterocyclyl may be unsubstituted or substituted, for instance 1 to 4 fold, e.g. 1 to 2 fold. Suitable substituents include e.g. halogen, halogenalkyl, alkyl, hydroxy, alkoxy, amino, alkylamino, dialkylamino, nitro, cyano, alkylsulfonamido, carboxy or heterocyclyl.

R3 in a compound of formula I may preferably denote hydrogen, lower alkyl, halogenalkyl or halogen, e.g. hydrogen, trifluoromethyl, fluoro or chloro.

R4 and R5 in a compound of formula I may preferably denote independently from each other hydrogen, alkyl or halogen, e.g. R4 and R5 denote both hydrogen, lower alkyl or halogen. Most preferably R4 and R5 both denote hydrogen.

A particularly preferred embodiment of the present invention is directed to a compound of formula

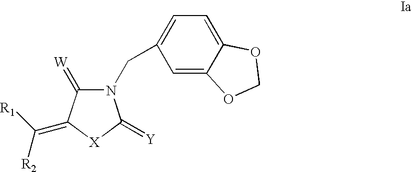

wherein R1, R2, W, X and Y are as defined above.

It will be apparent to one skilled in the art that certain compounds of this invention may exist in tautomeric forms. A compound according to formula I wherein X denotes NH and Y denotes O or S may exist in a lactam and a lactim form respectively in a thiolactam and thiolactim form. All such forms of the compounds according to formula I are understood to be within the scope of the present invention. Unless otherwise stated, structures described herein are also meant to include all stereochemical forms of the structure, e.g. the R/S or E/Z configurations.

Salts of the compounds of formula I, e.g. of formula Ia, include physiologically acceptable acid addition salts for example hydrochlorides, hydrobromides, sulphates, methane sulphonates, p-toluenesulphonates, phosphates, acetates, citrates, succinates, lactates, tartrates, fumarates and maleates. If a compound of formula I includes an acidic group, salts may also be formed with metal ions, for example alkali or earthalkaline salts such as sodium, potassium, magnesium, and calcium salts.

Compounds of formula I may be prepared from an intermediate compound of formula

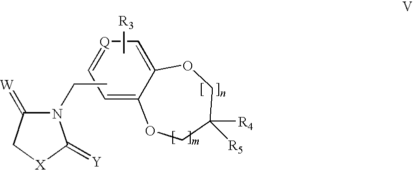

wherein W, X, Y, Q, R3, n, m, R4 and R5 are as defined above in formula I, by reacting a compound of formula V with an aldehyde or ketone of formula

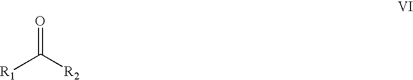

wherein R1 and R2 are defined as in formula I, to obtain a compound of formula I. The reaction of a compound of formula V with a compound of formula VI may be carried out in analogy to known methods, e.g. by Knoevenagel condensation, such as heating under reflux or microwave irradiating of a mixture of a compound of formula V with a compound of formula VI in the presence of trialkylamine, e.g. triethylamine, and an alkanol, e.g. ethanol. An alternative way for reacting a compound of formula V with a compound of formula VI may be by heating or microwave irradiation in the presence of an acetate salt and acetic acid analogously as for instance described in Orchard M. G. et al, Bioorg. Med. Chem. Lett. 2004, 14, 3975-3978.

A compound of formula V may be prepared in analogy to known methods, for instance a compound of formula V wherein X denotes NH and W, Y, Q, R3, R4 and R5 are as defined in formula I above may be prepared by cyclization of a compound of formula

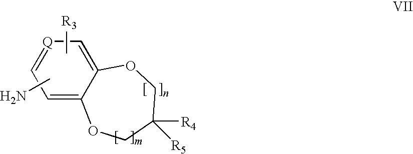

wherein Q, n, m, R4 and R5 have the same meaning as defined above in formula I with a compound of formula

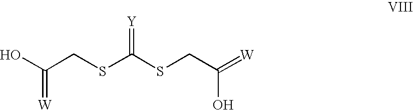

wherein Y and W are as defined above in formula I, to obtain a compound of formula V. This cyclization step may be carried out under conditions in analogy to, e.g. according to known processes, such as described in Woolhouse A. D. et al, J. Mater. Chem. 2001, 11, 2271-2281.

A compound of formula V wherein W, X, Z, Q, R3, n, m, R4 and R5 are as defined above in formula I may also be prepared by reacting a compound of formula

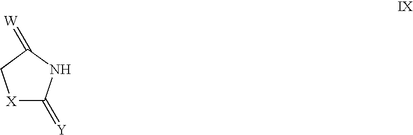

wherein W, X and Y are as defined above in formula I, with a compound of formula

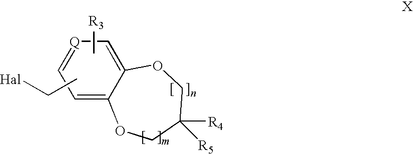

wherein R3, Q, n, m, R4 and R5 are as defined in formula I above and Hal denotes halogen, in order to obtain a compound of formula V. Such a reaction may be carried out under conditions in analogy, e.g. according to conventional preparation methods, e.g. by suspending a compound of formula IX in a suitable solvent, for instance an aprotic solvent such as DMSO, DMF or acetonitrile, and then adding a compound of formula X followed by stirring the suspension in the presence of a base such as DBU, alkali carbonates, alkali hydrides or alkali fluoride at room temperature. In order to isolate a compound of formula V from the reaction mixture the solvent may be removed by conventional methods, e.g. evaporation under reduced pressure, and a compound of formula V may optionally be purified by conventional methods, e.g. by column chromatography on silicagel.

Alternatively, a compound of formula V wherein X denotes S, O or NH and W, Y, Q, R3, n, m, R4 and R5 are as defined above in formula I may be prepared by reacting a compound of formula

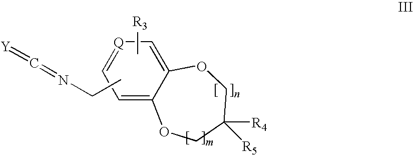

wherein Y, Q, R3, R4, R5, n and m have the same meaning as defined above in formula I, with a compound of formula

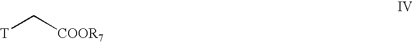

wherein R7 denotes alkyl and T denotes OH, SH or NH₂, e.g. under conventional conditions as described in Sim M. M. et al, Bioorg. Med. Chem. Lett. 2001, 11, 91-94.

A compound of formula I wherein X denotes CH₂ and W, Y, Q, R3, n, m, R4 and R5 are as defined above in formula I may also be prepared by reacting a compound of formula

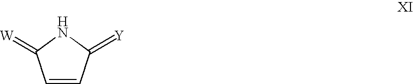

wherein W and Y have the same meaning as in formula I with a compound of formula

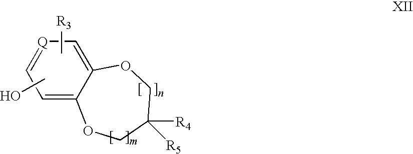

to obtain a compound of formula

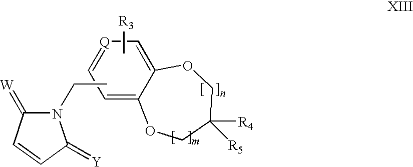

which may then be further reacted with a compound of formula

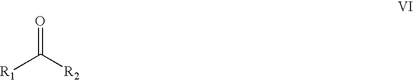

wherein R1 and R2 have the same meaning as defined above in formula I to obtain a compound of formula I wherein X denotes CH. The reaction of a compound of formula XI with a compound of formula XII may be carried out analogously to known methods, e.g. under conditions as known for Mitsunobu reactions, by adding di(lower alkyl)azodicarboxylate to a solution of a compound of formula XI and a compound of formula XII in a suitable solvent, e.g. THF, in the presence of triphenylphosphine and stirring that reaction mixture e.g. at room temperature, followed by removing the solvent, e.g. under reduced pressure by evaporation and finally purifying the obtained residue, e.g. by chromatography to isolate a compound of formula XIII.

The reaction of a compound of formula XIII with a compound of formula VI may also be carried out in analogy, e.g. according to known processes, for example by first heating a solution of a compound of formula XIII in the presence of triphenylphosphine in a suitable solvent, e.g. acetic acid, under reflux, removing the solvent and then suspending the obtained residue into a suitable solvent, e.g. toluene, in order to add a compound of formula VI and facilitating the reaction by increasing the temperature to e.g. 90-150° C. The obtained compound of formula I wherein X is CH may further optionally be purified by conventional methods, e.g. filtering, washing and drying.

A compound of formula VII wherein R3, n, m, R4 and R5 are as defined above in formula I may be prepared by cyclisation of a corresponding 3,4-dihydroxyaldehyde and a suitable ketone, dialkyl ketal or alkyl enolether under conditions known in the field (e.g. as described in Jae-Kon et. all. Bulletin of the Korean Chemical Society (2002), 23(5), 661-662; Yasuyuki Kita et all. JOC 1998, 63, 6625-6633; Reddy T J et al. JOC 2002, 67, 4635) followed by formation of an oxime by adding hydroxylamine in the presence of a base such as NaOH, triethylamine etc. (J. S. Buck, W. S. Ide. Organic Syntheses, Coll. Vol. 2, p. 622 (1943); Vol. 15, p. 85 (1935).) and reduction by catalytic hydrogenation or hydride complex reducing agents (DIBAL, LiAlH₄, NaBH₄, etc.) (M. Schlosser, J. Gorecka, E. Castagnetti. Eur. J. Org. Chem. 2003, 452-462).

Thus, in a further aspect the present invention provides a process for the preparation of a compound of formula I, e.g. a compound of formula Ia, as defined above comprising the steps of

a) reacting a compound of formula V as defined above with an aldehyde or ketone of formula IV as defined above to obtain a compound of formula I, and optionally
c) isolating a compound of formula I

In another aspect the present invention provides a process for the preparation of a compound of formula I wherein X denotes S, O or NH comprising the steps of

t) reacting a compound of formula III as defined above with a compound of formula VI as defined above to obtain a compound of formula V as defined above wherein X denotes S, O or NH,
b) reacting a compound of formula V as defined above with an aldehyde or ketone of formula IV as defined above to obtain a compound of formula I, and optionally
c) isolating a compound of formula I.

In another aspect the present invention relates to a process for the preparation of a compound of formula I as defined above wherein X denotes CH₂ comprising the steps of

i) reacting a compound of formula XI as defined above with a compound of formula XII as defined above to obtain a compound of formula XIII as defined above,
ii) reacting a compound of formula XIII as defined in step i) with a compound of formula VI wherein R1 and R2 have the same meaning as defined above in formula I to obtain a compound of formula I wherein X denotes CH, and optionally iii) isolating a compound of formula I wherein X denotes CH.

Step c), respectively step iii), the isolation of a compound of formula I, in the described processes may be carried out in analogy to, e.g. according to conventional methods for the isolation of compounds from reaction solutions, such as chromatography, filtration and/or drying.

The compounds according to formula I, e.g. those according to formula Ia, exhibit pharmacological activity and are therefore useful as pharmaceuticals. In particular, compounds of formula I show inhibitory activity against Mur ligases such as MurD and/or MurE. For instance, (Z)-3-(benzo[d][1,3]dioxol-5-yl-methyl)-5-(2-hydroxybenzylidene)-2-thioxothiazolidin-4-one (compound of Ex. 8) shows an IC₅₀ concentrations (inhibitory concentration required for 50% inhibition of enzyme activity) against MurE from S. aureus of 1.5 μM and (Z)-3-(benzo[d][1,3]dioxol-5-yl-methyl)-5-((4-oxo-2-((tetrahydrofuran-2-yl)methylamino)-4H-pyrido[1,2-a]pyrimidin-3-yl)methylene)-2-thioxothiazolidin-4-one (compound of Ex. 3) at 100 μM concentration leads to a residual activity of MurD from E. coli of 7% when tested in an MurD enzyme assay.

Mur ligases (MurC, MurD, MurE, MurF) catalyse the stepwise addition of L-alanine, D-glutamic acid, a diamino acid (usually diaminopimelic acid or L-lysine), and a dipeptide D-alanine-X (wherein X is usually D-alanine and less frequently X is D-lactate or D-serine) onto the D-lactoyl group of cytoplasmic peptidoglycan precursor UDP-MurNAc. (J. A. Bertrand, et al., Journal of Molecular Biology, 1999, 289, 579-590)

Among Mur family enzymes, UDP-N-acetylmuramoyl-L-alanin:D-glutamate ligase (MurD) appears to represent a very good target for an antibiotic agent due to the presence of L-Ala-D-Glu link in every eubacterial cell. In addition, D-amino acid residues cannot be found in eukaryotic cells. Therefore, MurD inhibitors such as the compounds of formula I according to the present invention may possess very good properties with respect to selective toxicity.

MurE synthetase catalyses the addition of either meso-diaminopimelic acid or L-lysine to the substrate UDP-N-acetylmuramoyl-L-alanin:D-glutamate (depending on the organism). Rarely, an ornithine, diaminobutyric acid, homoserine, lanthionine, or 3-hydroxy-2,6-diaminopimelic acid is located at position 3 in the peptide subunit (J. Van Heijenoort, Natural Product Reports, 2001, 18, 503-519). In general, the name MurE stands for UDP-N-acetylmuramoyl-L-alanin-D-glutamate:meso-diaminopimeate ligase or UDP-N-acetylmuramoyl-L-alanin-D-glutamate:L-lysine ligase, depending which residue the enzyme adds to the growing peptide chain.

In another aspect the present invention provides thus a compound of formula I, e.g. of formula Ia, as a pharmaceutical, preferably as an antimicrobial agent, such as an antibiotic.

In a further aspect the present invention provides a compound of formula I, e.g. of formula Ia for use in the preparation of a medicament for the treatment of microbial diseases,

In a further aspect the present invention is directed to the use of a compound of formula I in the preparation of a medicament for the treatment of microbial diseases.

The term microbial diseases includes diseases caused by bacteriae having Mur ligases, e.g. having MurD and/or MurE ligases such as those bacteria which are sensitive to MurD inhibitors and/or MurE inhibitors for example bacteria of strains E. coli, E. faecalis S. aureus including MRSA strains.

In a further aspect the present invention provides a method of treatment of microbial, e.g. bacterial, diseases which comprises administering to a subject in need of such treatment an effective amount of a compound of formula I.

The appropriate dosage and treatment regimens for any particular patient will depend upon a variety of factors, including the activity of the specific compound employed, the age, body weight, general health status, sex, diet, time of administration, route of administration, rate of excretion, drug combination, the severity and course of the disease, and the patient's disposition to the disease and the judgment of the treating physician. However, in general, for satisfactory results in larger mammals, for example humans, an indicated daily dosage is in the range from about 0.05 to 5 g, for example 0.1 to about 2.5 g, of a compound of formula I, conveniently administered, for example, in divided doses up to four times a day. Safe and effective dosages for different classes of patients and for different disease states will be determined by clinical trial as is required in the art.

In another aspect the present invention provides a pharmaceutical composition comprising a compound of formula I, e.g. of formula Ia, or a salt thereof. Beside a compound of formula I a pharmaceutical composition according to the present invention may contain one or more pharmaceutical acceptable excipient(s), such as carriers or diluents and optionally additional therapeutically effective compounds. A pharmaceutical composition according to the present invention may be useful for treating bacterial infections.

The term pharmaceutically acceptable excipient refers to conventional excipients as known in the pharmaceutical field, e.g. a non-toxic carrier that may be administered to a patient, together with a compound of formula I, and which does not destroy the pharmacological activity thereof.

The pharmaceutical compositions of this invention may be administered orally, parenterally, by inhalation spray, topically, via ophthalmic solution or ointment, rectally, nasally, buccally, vaginally or via implanted reservoir. The term parenteral as used herein includes subcutaneous, intracutaneous, intravenous, intramuscular, intra-articular, intrasynovial, intrasternal, intrathecal, intralesional and intracranial injection or infusion techniques.

Orally administrable compositions may be for instance in the form of tablets, capsules, powders or granules. Parenterally administrable pharmaceutical compositions may be for instance a suspension or solution of a compound of formula I in a sterile liquid.

The pharmaceutical compositions according to the present invention may be prepared in analogy to, e.g. according to known, conventional methods and techniques in the pharmaceutical field, e.g. analogously to the application of known antibiotics such as Fluorochinolones, e.g. Ciprofloxacin or Enrofloxacin.

Compounds of this invention may be employed in a conventional manner for controlling bacterial infections and for treating diseases or reducing the advancement or severity of effects, which are mediated by bacteria in mammals, e.g. humans or animals. Such methods of treatment, their dosage levels and requirements may be selected by those of ordinary skill in the art from available methods and techniques.

While we have described a number of examples of this invention, it is apparent that our basic constructions may be altered to provide other embodiments which utilize the compounds, processes and compositions of this invention

### EXAMPLES

### General Procedure A for Synthesis of 3-(benzo[d][1,3]-dioxol-5-ylmethyl)rhodanines

### Step 1. Preparation of 3-(benzo[d][1,3]dioxol-5-ylmethyl)-2-thioxothiazolidin-4-one

A suspension of piperonylamine (16.5 mmol, 2.50 g) in 1M NaOH is refluxed for 10 min and cooled to room temperature. Subsequently bis(carboxymethyl) trithiocarbonate (3.74 g, 16.5 mmol) is added and the reaction mixture refluxed overnight to give the precipitate, which is filtered off and ished with water (3×20 mL). The remaining residue is suspended in a mixture of methanol and isopropanol (1:1, 25 mL) and refluxed for 10 minutes. Cooled reaction mixture is filtered and the solid residue dried at lower temperature and recrystallyzed from methanol to give 3.12 g of 3-(benzo[d][1,3]dioxol-5-ylmethyl)-2-thioxothiazolidin-4-one.

### Step 2. Coupling of Aldehydes or Ketones to 3-(benzo[d][1,3]dioxol-5-ylmethyl)-2-thioxothiazolidin-4-one

### Alternative a) Coupling Procedure Using Triethylamine and Ethanol

A solution of 3-(benzo[d][1,3]dioxol-5-ylmethyl)-2-thioxothiazolidin-4-one (100 mg, 0.374 mmol), corresponding aldehyde R₁CHO or ketone R₁(CO)R₂ (0.374 mmol) and triethylamine (0.104 mL, 0.748 mmol) in ethanol (3 mL) is heated in the microwave reactor for 2 h. The reaction mixture is cooled to room temperature and the obtained precipitate filtered off and washed with EtOH (2×5 mL) and MeOH (2×5 mL) and dried at elevated temperature.

### Alternative b) Coupling Procedure Using Sodium Acetate and Acetic Acid

A solution of 80 mg (=0.299 mmol) 3-(benzo[d][1,3]dioxol-5-ylmethyl)-2-thioxothiazolidin-4-one, corresponding aldehyde R1HCO (0.299 mmol) and sodium acetate (127.4 mg, 1.50 mmol) in acetic acid (3 mL) is heated in the microwave reactor for 2 h at around 130° C. The reaction mixture is cooled to room temperature and the obtained precipitate filtered off and washed with EtOH (2×5 mL) and MeOH (2×5 mL) and dried at elevated temperature.

### General Procedure B for Synthesis of 3-((2,2-dimethylbenzo[d][1,3]dioxol-5-yl)methyl)rhodanines

### Step 1. Synthesis of 2,2-dimethylbenzo[d][1,3]dioxole-5-carbaldehyde

Phosphorus pentoxide (226 mmol, 32.1 g) is added into a solution of 3,4-dihydroxybenzaldehyde (113 mmol, 15.6 g) and acetone (16.6 mL, 226 mmol) in toluene (230 mL) and stirred at room temperature for 2 hours. Subsequently additional acetone (25 mL) and phosphorus pentoxide (106 mmol, 15.0 g) are added. The reaction mixture is stirred for 2.5 h at room temperature and then poured into a solution of potassium carbonate (140 g) in H₂O (1 L). Ethylacetate is added and the fractions separated. The organic fraction is washed with saturated solution of NaCl, dried over MgSO₄ and the solvent removed under reduced pressure. The obtained oily residue is distilled under reduced pressure to yield 2.2 g (11%) of 2,2-dimethylbenzo[d][1,3]dioxole-5-carbaldehyde.

### Step 2. Synthesis of 2,2-dimethylbenzo[d][1,3]dioxole-5-carbaldehyde oxime

A solution of sodium hydroxide (18.5 mmol, 0.741 g) in H₂O is added into a solution of 2,2-dimethylbenzo[d][1,3]dioxole-5-carbaldehyde (12.4 mmol, 2.20 g) and hydroxylamine hydrochloride (14.8 mmol, 1.03 g) in ethanol (50 mL) and the reaction mixture stirred overnight. Subsequently diethylether (50 mL) is added and washed with saturated solution of NaCl (2×30 mL), dried with MgSO₄ and the solvent removed under reduced pressure. The product is washed with hexane to yield 1.87 g (78%) of 2,2-dimethylbenzo[d][1,3]dioxole-5-carbaldehyde oxime.

### Step 3. Synthesis of (2,2-dimethylbenzo[d][1,3]dioxol-5-yl)methanamine

2,2-Dimethylbenzo[d][1,3]dioxole-5-carbaldehyde oxime (8.85 mmol, 1.71 g) is added into a suspension of LiAlH₄ (17.7 mmol, 0.672 g) in dry diethylether (25 mL) and refluxed for 4 hours. After the reaction is complete ethylacetate (60 mL) and water (10 mL) were slowly added into the reaction mixture. The fractions were separated and the organic fraction is additionally washed with saturated solution of NaCl (30 mL) and dried with MgSO₄. The solvent is removed under reduced pressure and the oily residue purified with column chromatography (silicagel; ethylacetate/methanol/triethylamine=1/1/0.01) to yield 0.90 g (57%) of (2,2-dimethylbenzo[d][1,3]dioxol-5-yl)methanamine.

### Step 4. Synthesis of 3-((2,2-dimethylbenzo[d][1,3]dioxol-5-yl)methyl)-2-thioxo-thiazolidin-4-one

Synthesis of 3-((2,2-dimethylbenzo[d][1,3]dioxol-5-yl)methyl)-2-thioxothiazolidin-4-one is performed in a similar manner as described in example 62.

### Step 5. Coupling of aldehydes to 3-((2,2-dimethylbenzo[d][1,3]dioxol-5-yl)methyl)-2-thioxothiazolidin-4-one

Coupling with aldehydes of formula R₁CHO to 3-((2,2-dimethylbenzo[d][1,3]dioxol-5-yl)methyl)-2-thioxothiazolidin-4-one is performed in a similar manner as described in General Procedure A.

### General Procedure C for synthesis of 5-substituted 3-((6-chlorobenzo[d][1,3]dioxol-5-yl)methyl)-2-thioxothiazolidin-4-one

### Step 1. Synthesis of 3-((6-chlorobenzo[d][1,3]dioxol-5-yl)methyl)-2-thioxothiazolidin-4-one

A suspension of thiazolidine-2,4-dione (5.0 mmol, 0.586 g), 5-chloro-6-(chloromethyl)benzo[d][1,3]dioxole (5.0 mmol, 1.03 g) and potassium fluoride (5.0 mmol, 0.291 g) in DMF (10 mL) is stirred at room temperature overnight. The solvent is removed under reduced pressure and the residue purified by column chromatography (silicagel; ethylacetate/hexane=¼) to give 0.649 g (45%) of 3-((6-chlorobenzo[d][1,3]dioxol-5-yl)methyl)-2-thioxothiazolidin-4-one.

### Step 2. Coupling of aldehydes to 3-((6-chlorobenzo[d][1,3]dioxol-5-yl)methyl)-2-thioxothiazolidin-4-one

Coupling of aldehydes to 3-((6-chlorobenzo[d][1,3]dioxol-5-yl)methyl)-2-thioxothiazolidin-4-one is performed in a similar manner as described in General Procedure A.

TABLE 1
The compounds of Examples 1 to 50 and 55 to 61 are prepared according to the
General Procedure A and compounds of Examples 51 to 52 according to General Procedure
B as set out above from reactions with corresponding aldehydes of formula R₁HCO.
Compound of Example 54 is prepared according to process of Example 62; compound of
Example 55 is prepared according to General Procedure C as described above

| Ex. | | | |
|---|---|---|---|
| No. | R₁ | Structure | Name |
| 1 | 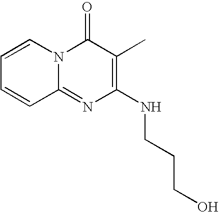 | 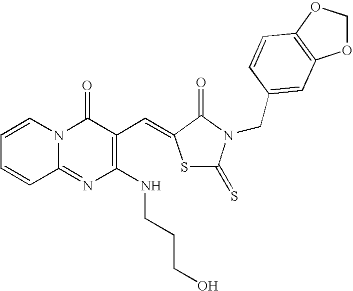 | (Z)-3-(benzo[d][1,3]dioxol-5-yl-methyl)- 5-((2-(3-hydroxypropylamino)-4-oxo- 4H-pyrido[1,2-a]pyrimidin-3- yl)methylene)-2-thioxothiazolidin-4-one |
| 2 | 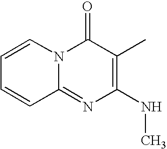 | 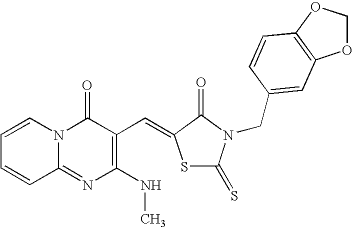 | (Z)-3-(benzo[d][1,3]dioxol-5-yl-methyl)- 5-((2-(methylamino)-4-oxo-4H- pyrido[1,2-a]pyrimidin-3-yl)methylene)- 2-thioxothiazolidin-4-one |
| 3 | 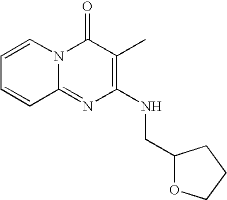 | 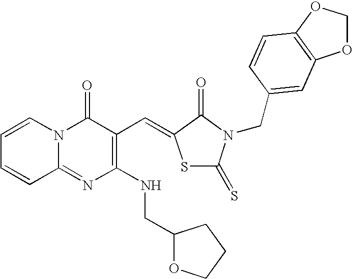 | (Z)-3-(benzo[d][1,3]dioxol-5-yl-methyl)- 5-((4-oxo-2-((tetrahydrofuran-2- yl)methylamino)-4H-pyrido[1,2- a]pyrimidin-3-yl)methylene)-2- thioxothiazolidin-4-one |
| 4 | 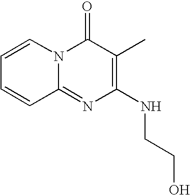 | 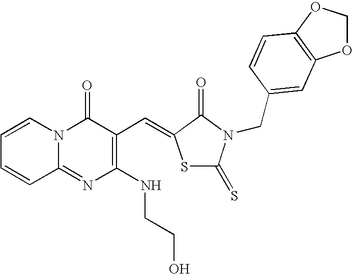 | (Z)-3-(benzo[d][1,3]dioxol-5-yl-methyl)- 5-((2-(2-hydroxyethylamino)-4-oxo-4H- pyrido[1,2-a]pyrimidin-3-yl)methylene)- 2-thioxothiazolidin-4-one |
| 5 | 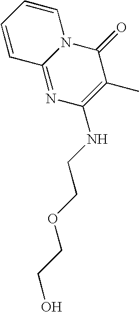 | 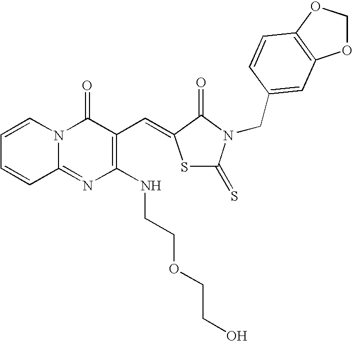 | (Z)-3-(benzo[d][1,3]dioxol-5-yl-methyl)- 5-((2-(2-(2-hydroxyethoxy)ethylamino)- 4-oxo-4H-pyrido[1,2-a]pyrimidin-3- yl)methylene)-2-thioxothiazolidin-4-one |
| 6 | 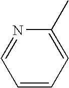 | 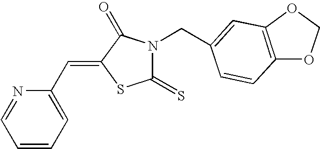 | (Z)-3-(benzo[d][1,3]dioxol-5-yl-methyl)- 5-(pyridin-2-yl-methylene)-2- thioxothiazolidin-4-one |
| 7 | 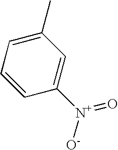 | 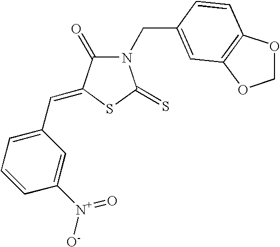 | (Z)-3-(benzo[d][1,3]dioxol-5-yl-methyl)- 5-(3-nitrobenzylidene)-2- thioxothiazolidin-4-one |
| 8 | 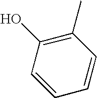 | 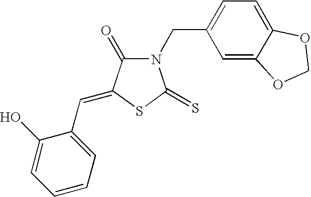 | (Z)-3-(benzo[d][1,3]dioxol-5-yl-methyl)- 5-(2-hydroxybenzylidene)-2- thioxothiazolidin-4-one |
| 9 | 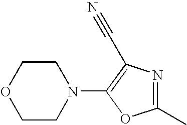 | 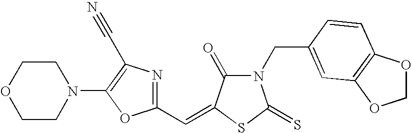 | (E)-2-((3-(benzo[d][1,3]dioxol-5-yl- methyl)-4-oxo-2-thioxothiazolidin-5- ylidene)methyl)-5-morpholinooxazole- 4-carbonitrile |
| 10 | 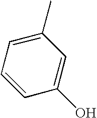 | 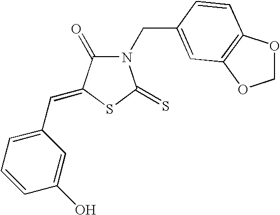 | (Z)-3-(benzo[d][1,3]dioxol-5-yl-methyl)- 5-(3-hydroxybenzylidene)-2- thioxothiazolidin-4-one |
| 11 | 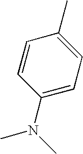 | 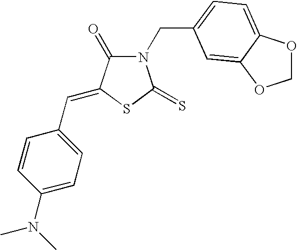 | (Z)-3-(benzo[d][1,3]dioxol-5-yl-methyl)- 5-(4-(dimethylamino)benzylidene)-2- thioxothiazolidin-4-one |
| 12 | 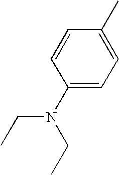 | 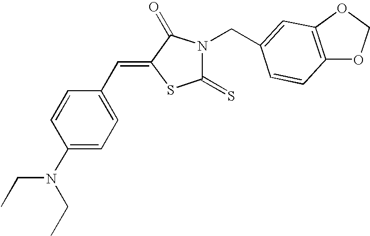 | (Z)-3-(benzo[d][1,3]dioxol-5-yl-methyl)- 5-(4-(diethylamino)benzylidene)-2- thioxothiazolidin-4-one |
| 13 | 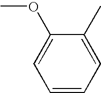 | 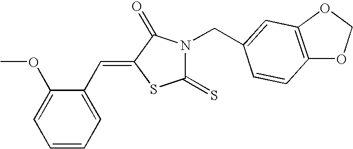 | (Z)-3-(benzo[d][1,3]dioxol-5-yl-methyl)- 5-(2-methoxybenzylidene)-2- thioxothiazolidin-4-one |
| 14 | 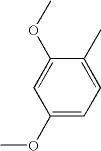 | 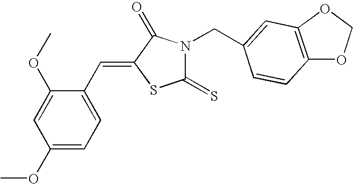 | (Z)-3-(benzo[d][1,3]dioxol-5-yl-methyl)- 5-(2,4-dimethoxybenzylidene)-2- thioxothiazolidin-4-one |
| 15 | 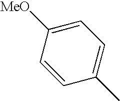 | 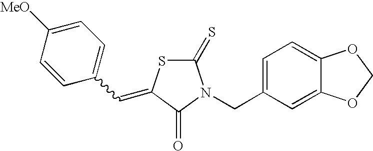 | 3-(benzo[d][1,3]dioxol-5-ylmethyl)-5-(4- methoxybenzylidene)-2- thioxothiazolidin-4-one |
| 16 | 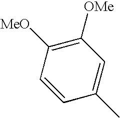 | 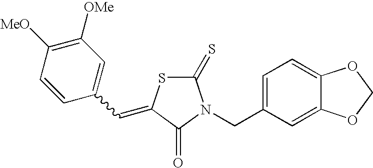 | 3-(benzo[d][1,3]dioxol-5-ylmethyl)-5- (3,4-dimethoxybenzylidene)-2- thioxothiazolidin-4-one |
| 17 | 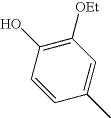 | 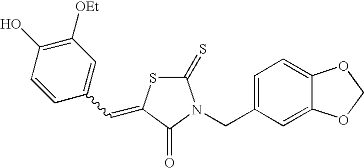 | 3-(benzo[d][1,3]dioxol-5-ylmethyl)-5-(3- ethoxy-4-hydroxybenzylidene)-2- thioxothiazolidin-4-one |
| 18 | 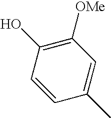 | 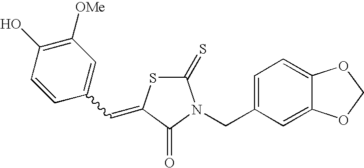 | 3-(benzo[d][1,3]dioxol-5-ylmethyl)-5-(4- hydroxy-3-methoxybenzylidene)-2- thioxothiazolidin-4-one |
| 19 | 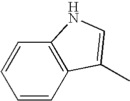 | 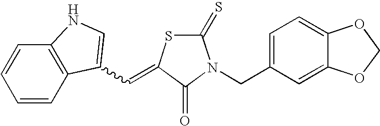 | 5-((1H-indol-3-yl)methylene)-3- (benzo[d][1,3]dioxol-5-ylmethyl)-2- thioxothiazolidin-4-one |
| 20 | 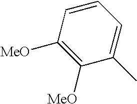 | 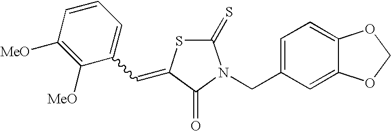 | 3-(benzo[d][1,3]dioxol-5-ylmethyl)-5- (2,3-dimethoxybenzylidene)-2- thioxothiazolidin-4-one |
| 21 | 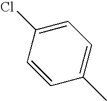 | 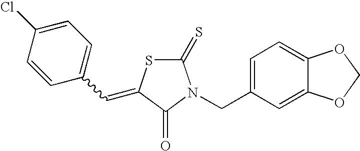 | 3-(benzo[d][1,3]dioxol-5-ylmethyl)-5-(4- chlorobenzylidene)-2-thioxothiazolidin- 4-one |
| 22 | 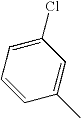 | 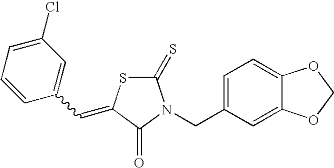 | 3-(benzo[d][1,3]dioxol-5-ylmethyl)-5-(3- chlorobenzylidene)-2-thioxothiazolidin- 4-one |
| 23 | 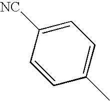 | 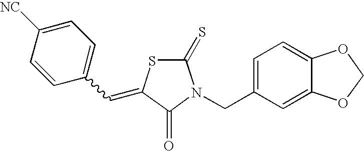 | 4-((3-(benzo[d][1,3]dioxol-5-ylmethyl)- 4-oxo-2-thioxothiazolidin-5- ylidene)methyl)benzonitrile |
| 24 | 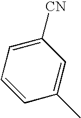 | 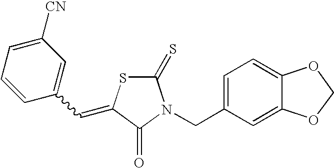 | 3-((3-(benzo[d][1,3]dioxol-5-ylmethyl)- 4-oxo-2-thioxothiazolidin-5- ylidene)methyl)benzonitrile |
| 25 | 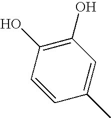 | 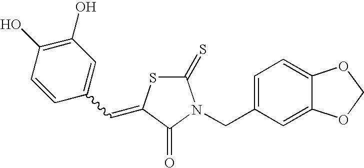 | 3-(benzo[d][1,3]dioxol-5-ylmethyl)-5- (3,4-dihydroxybenzylidene)-2- thioxothiazolidin-4-one |
| 26 | 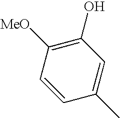 | 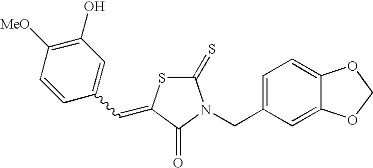 | 3-(benzo[d][1,3]dioxol-5-ylmethyl)-5-(3- hydroxy-4-methoxybenzylidene)-2- thioxothiazolidin-4-one |
| 27 | 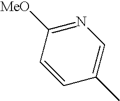 | 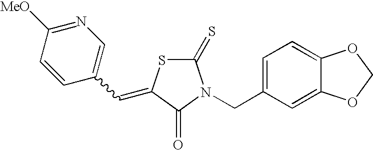 | 3-(benzo[d][1,3]dioxol-5-ylmethyl)-5- ((6-methoxypyridin-3-yl)methylene)-2- thioxothiazolidin-4-one |
| 28 | 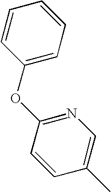 | 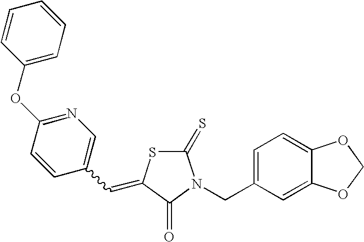 | 3-(benzo[d][1,3]dioxol-5-ylmethyl)-5- ((6-phenoxypyridin-3-yl)methylene)-2- thioxothiazolidin-4-one |
| 29 | 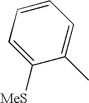 | 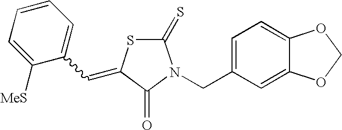 | 3-(benzo[d][1,3]dioxol-5-ylmethyl)-5-(2- (methylthio)benzylidene)-2- thioxothiazolidin-4-one |
| 30 | 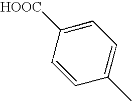 | 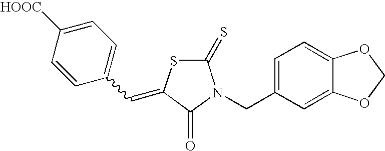 | 4-((3-(benzo[d][1,3]dioxol-5-ylmethyl)- 4-oxo-2-thioxothiazolidin-5- ylidene)methyl)benzoic acid |
| 31 | 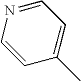 | 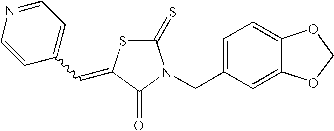 | 3-(benzo[d][1,3]dioxol-5-ylmethyl)-5- (pyridin-4-ylmethylene)-2- thioxothiazolidin-4-one |
| 32 | 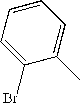 | 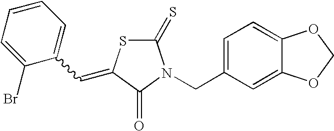 | 3-(benzo[d][1,3]dioxol-5-ylmethyl)-5-(2- bromobenzylidene)-2-thioxothiazolidin- 4-one |
| 33 | 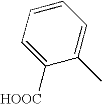 | 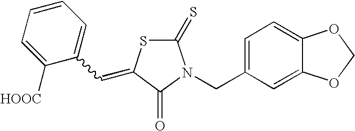 | 2-((3-(benzo[d][1,3]dioxol-5-ylmethyl)- 4-oxo-2-thioxothiazolidin-5- ylidene)methyl)benzoic acid |
| 34 | 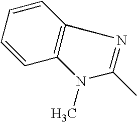 | 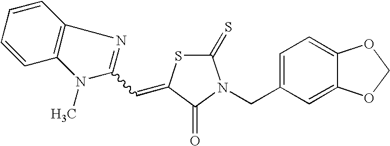 | 3-(benzo[d][1,3]dioxol-5-ylmethyl)-5- ((1-methyl-1H-benzo[d]imidazol-2- yl)methylene)-2-thioxothiazolidin-4-one |
| 35 | 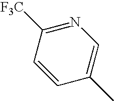 | 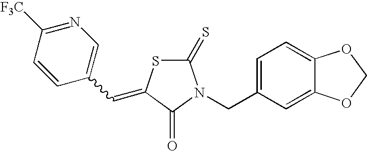 | 3-(benzo[d][1,3]dioxol-5-ylmethyl)-2- thioxo-5-((6-(trifluoromethyl)pyridin-3- yl)methylene)thiazolidin-4-one |
| 36 | 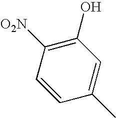 | 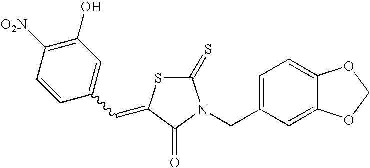 | 3-(benzo[d][1,3]dioxol-5-ylmethyl)-5-(3- hydroxy-4-nitrobenzylidene)-2- thioxothiazolidin-4-one |
| 37 | 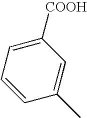 | 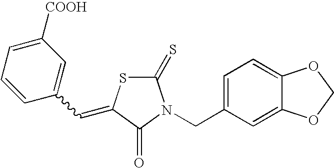 | 3-((3-(benzo[d][1,3]dioxol-5-ylmethyl)- 4-oxo-2-thioxothiazolidin-5- ylidene)methyl)benzoic acid |
| 38 | 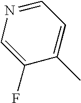 | 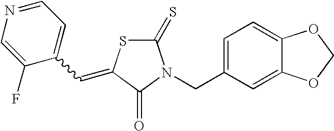 | 3-(benzo[d][1,3]dioxol-5-ylmethyl)-5- ((3-fluoropyridin-4-yl)methylene)-2- thioxothiazolidin-4-one |
| 39 | 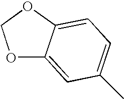 | 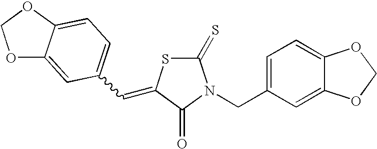 | 3-(benzo[d][1,3]dioxol-5-ylmethyl)-5- (benzo[d][1,3]dioxol-5-ylmethylene)-2- thioxothiazolidin-4-one |
| 40 | 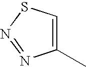 | 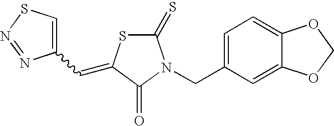 | 5-((1,2,3-thiadiazol-4-yl)methylene)-3- (benzo[d][1,3]dioxol-5-ylmethyl)-2- thioxothiazolidin-4-one |
| 41 | 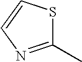 | 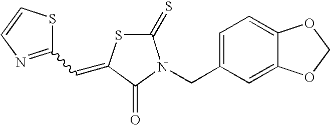 | 3-(benzo[d][1,3]dioxol-5-ylmethyl)-5- (thiazol-2-ylmethylene)-2- thioxothiazolidin-4-one |
| 42 | 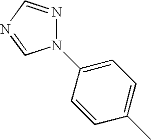 | 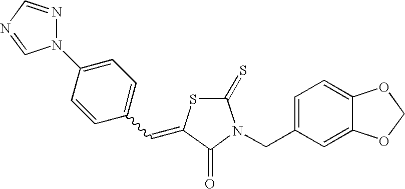 | 5-(4-(1H-1,2,4-triazol-1- yl)benzylidene)-3-(benzo[d][1,3]dioxol- 5-ylmethyl)-2-thioxothiazolidin-4-one |
| 43 | 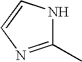 | 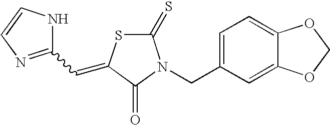 | 5-((1H-imidazol-2-yl)methylene)-3- (benzo[d][1,3]dioxol-5-ylmethyl)-2- thioxothiazolidin-4-one |
| 44 | 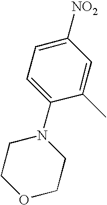 | 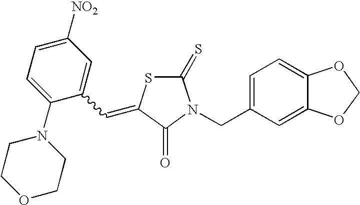 | 3-(benzo[d][1,3]dioxol-5-ylmethyl)-5-(2- morpholino-5-nitrobenzylidene)-2- thioxothiazolidin-4-one |
| 45 | 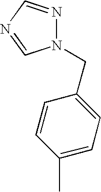 | 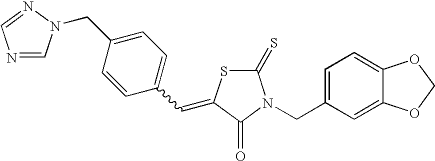 | 5-(4-((1H-1,2,4-triazol-1- (benzo[d][1,3]dioxol-5-ylmethyl)-2- thioxothiazolidin-4-one |
| 46 | 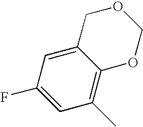 | 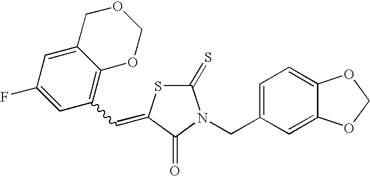 | 3-(benzo[d][1,3]dioxol-5-ylmethyl)-5- ((6-fluoro-4 H-benzo[d][1,3]dioxin-8- yl)methylene)-2-thioxothiazolidin-4-one |
| 47 | 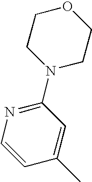 | 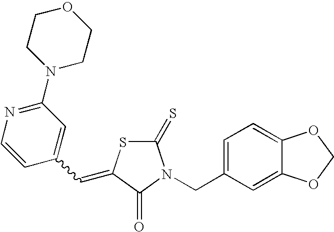 | 3-(benzo[d][1,3]dioxol-5-ylmethyl)-5- ((2-morpholinopyridin-4-yl)methylene)- 2-thioxothiazolidin-4-one |
| 48 | 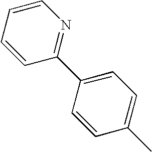 | 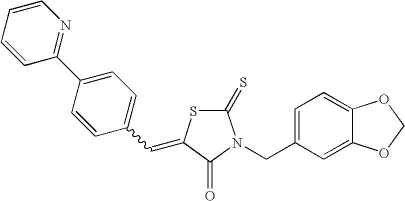 | 3-(benzo[d][1,3]dioxol-5-ylmethyl)-5-(4- (pyridin-2-yl)benzylidene)-2- thioxothiazolidin-4-one |
| 49 | 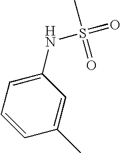 | 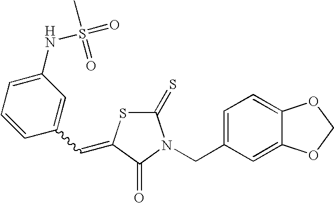 | N-(3-((3-(benzo[d][1,3]dioxol-5- ylmethyl)-4-oxo-2-thioxothiazolidin-5- ylidene)methyl)phenyl)methanesulfon- amide |
| 50 | 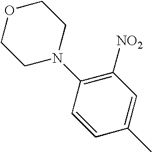 | 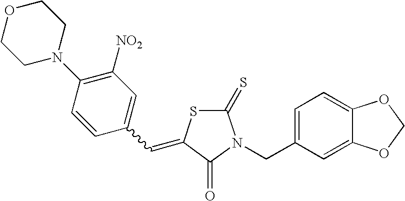 | 3-(benzo[d][1,3]dioxol-5-ylmethyl)-5-(4- morpholino-3-nitrobenzylidene)-2- thioxothiazolidin-4-one |
| 51 | 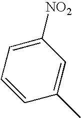 | 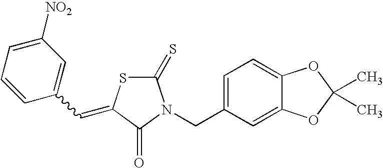 | 3-((2,2-dimethylbenzo[d][1,3]dioxol-5- yl)methyl)-5-(3-nitrobenzylidene)-2- thioxothiazolidin-4-one |
| 52 | 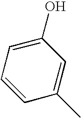 | 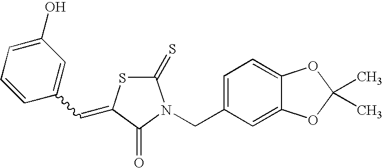 | 3-((2,2-dimethylbenzo[d][1,3]dioxol-5- yl)methyl)-5-(3-hydroxybenzylidene)-2- thioxothiazolidin-4-one |
| 53 | 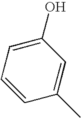 | 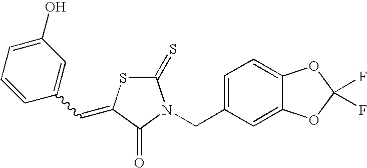 | 3-((2,2-difluorobenzo[d][1,3]dioxol-5- yl)methyl)-5-(3-hydroxybenzylidene)-2- thioxothiazolidin-4-one |
| 54 | 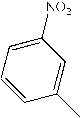 | 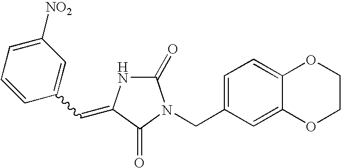 | 3-((2,3-dihydrobenzo[b][1,4]dioxin-6- yl)methyl)-5-(3- nitrobenzylidene)imidazolidine-2,4- dione |
| 55 | 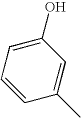 | 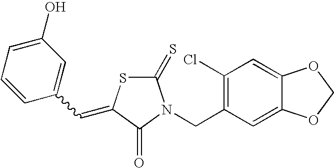 | 3-((6-chlorobenzo[d][1,3]dioxol-5- yl)methyl)-5-(3-hydroxybenzylidene)-2- thioxothiazolidin-4-one |
| 56 | 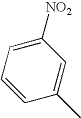 | 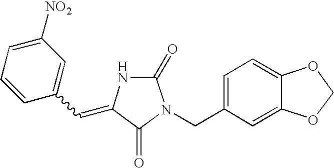 | 3-(benzo[d][1,3]dioxol-5-ylmethyl)-5-(3- nitrobenzylidene)imidazolidine-2,4- dione |
| 57 | 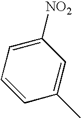 | 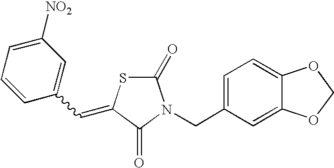 | 3-(benzo[d][1,3]dioxol-5-ylmethyl)-5-(3- nitrobenzylidene)thiazolidine-2,4-dione |
| 58 | 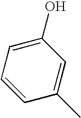 | 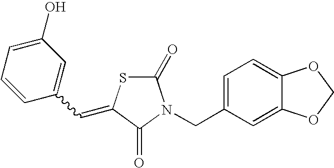 | 3-(benzo[d][1,3]dioxol-5-ylmethyl)-5-(3- hydroxybenzylidene)thiazolidine-2,4- dione |
| 59 | 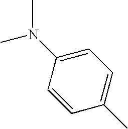 | 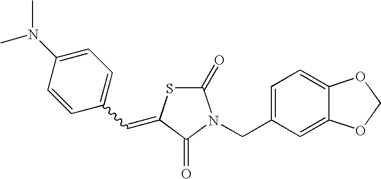 | 3-(benzo[d][1,3]dioxol-5-ylmethyl)-5-(4- (dimethylamino)benzylidene)thiazolidine- 2,4-dione |
| 60 | 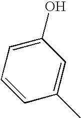 | 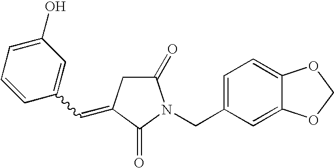 | 1-(benzo[d][1,3]dioxol-5-ylmethyl)-3-(3- hydroxybenzylidene)pyrrolidine-2,5- dione |
| 61 | 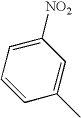 | 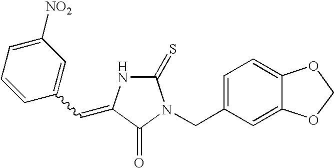 | 3-(benzo[d][1,3]dioxol-5-ylmethyl)-5-(3- nitrobenzylidene)-2-thioxoimidazolidin- 4-one |

Analytical data for abovementioned examples:

### Example 1

¹H NMR (300 MHz, DMSO-d₆) δ 1.76 (2H, q, J=6.5 Hz), 3.49 (2H, m), 3.55 (2H, m), 4.52 (1H, t, J=5.1 Hz), 5.13 (2H, s), 5.99 (2H, s), 6.80-7.00 (3H, m), 7.13 (1H, tm, J=6.9 Hz), 7.34 (1H, d, J=8.9), 7.74 (1H, s), 7.89 (1H, m), 8.16 (1H, t, J=5.4 Hz),), 8.76 (1H, d, J=7.1 Hz); EI MS m/z 496 [M+], FAB MS m/z 497 [MH+]

### Example 2

¹H NMR (300 MHz, DMSO-d₆) δ 2.97 (2H, d, J=4.4), 5.14 (2H, s), 5.99 (2H, s), 6.80-6.96 (3H, m), 7.14 (1H, tm, J=6.9 Hz), 7.37 (1H, d, J=8.9), 7.73 (1H, s), 7.90 (1H, m), 8.15 (1H, m), 8.78 (1H, d, J=7.3 Hz); EI MS m/z 452 [M+], FAB MS m/z 453 [MH+]

### Example 3

¹H NMR (300 MHz, DMSO-d₆) δ 1.63 (1H, m), 1.86 (3H, m), 3.44-3.70 (3H, m), 3.80 (1H, m), 4.10 (1H, m), 5.14 (2H, s), 5.99 (2H, s), 6.80-7.00 (3H, m), 7.14 (1H, tm, J=7.0 Hz), 7.34 (1H, d, J=8.8), 7.75 (1H, s), 7.89 (1H, m), 8.28 (1H, t, J=5.5), 8.77 (1H, d, J=7.0 Hz); EI MS m/z 522 [M+], FAB MS m/z 523 [MH+]

### Example 4

¹H NMR (300 MHz, DMSO-d₆) δ 3.58 (4H, m), 4.78 (1H, m), 5.14 (2H, s), 5.99 (2H, s), 6.80-6.96 (3H, m), 7.13 (1H, tm, J=6.9 Hz), 7.34 (1H, d, J=8.9), 7.76 (1H, s), 7.89 (1H, m), 8.15 (1H, m), 8.77 (1H, dm, J=7.0 Hz); EI MS m/z 482 [M+], FAB MS m/z 483 [MH+]

### Example 5

¹H NMR (300 MHz, DMSO-d₆) δ 3.42-3.75 (8H, m), 4.59 (1H, m), 5.14 (2H, s), 5.99 (2H, s), 6.80-6.96 (3H, m), 7.14 (1H, td, J=7.0 Hz, 1.1 Hz), 7.36 (1H, d, J=8.8), 7.74 (1H, s), 7.90 (1H, m), 8.22 (1H, t, J=5.0 Hz), 8.78 (1H, d, J=7.1 Hz); EI MS m/z 526 [M+], FAB MS m/z 527 [MH+]

### Example 6

¹H NMR (300 MHz, DMSO-d₆) δ 5.15 (2H, s), 5.99 (2H, s), 6.79-6.95 (3H, m), 7.46 (1H, m), 7.87 (1H, s), 7.97 (2H, m), 8.81 (1H, d, J=4.5 Hz); EI MS m/z 356 [M+], FAB MS m/z 357 [MH+]

### Example 7

¹H NMR (300 MHz, DMSO-d₆) δ 5.16 (2H, s), 6.00 (2H, s), 6.80-6.98 (3H, m), 7.85 (1H, t, J=8.0 Hz), 8.03 (1H, s), 8.06 (1H, dm, J=8.1 Hz), 8.33 (1H, dm, J=8.2 Hz), 8.52 (1H, m); EI MS m/z 400 [M+], FAB MS m/z 401 [MH+]

### Example 8

¹H NMR (300 MHz, DMSO-d₆) δ 5.13 (2H, s), 5.98 (2H, s), 6.78-7.02 (5H, m), 7.30-7.40 (2H, m), 8.01 (1H, s), 10.77 (1H, s); EI MS m/z 371 [M+], FAB MS m/z 372 [MH+]

### Example 9

¹H NMR (300 MHz, DMSO-d₆) δ 3.60 (4H, m), 3.77 (4H, m), 5.11 (2H, s), 5.99 (2H, s), 6.78-6.89 (2H, m), 6.91 (1H, s), 6.78-6.94 (3H, m), 7.32 (1H, s); EI MS m/z 456 [M+], FAB MS m/z 457 [MH+]

### Example 10

¹H NMR (300 MHz, DMSO-d₆) δ 5.14 (2H, s), 5.99 (2H, s), 6.80-6.96 (4H, m), 7.02 (1H, t, J=1.9 Hz), 7.10 (1H, d, J=7.9 Hz), 7.36 (1H, d, J=7.9 Hz), 7.75 (1H, s), 9.90 (1H, s); EI MS m/z 371 [M+], FAB MS m/z 372 [MH+]

### Example 11

¹H NMR (300 MHz, DMSO-d₆) δ 3.04 (6H, s), 5.12 (2H, s), 5.98 (2H, s), 6.75-6.95 (5H, m), 7.48 (2H, m), 7.71 (1H, s); EI MS m/z 398 [M+], FAB MS m/z 399 [MH+]

### Example 12

¹H NMR (300 MHz, DMSO-d₆) δ 1.13 (6H, t, J=7.0 Hz), 3.45 (4H, q, J=7.1 Hz), 5.13 (2H, s), 5.99 (2H, s), 6.78-6.94 (5H, m), 7.46 (2H, m), 7.69 (1H, s); FAB MS m/z 427 [MH+]

### Example 13

¹H NMR (300 MHz, DMSO-d₆) δ 3.91 (3H, s), 5.14 (2H, s), 5.99 (2H, s), 6.80-6.89 (2H, m), 6.92 (1H, d, J=1.3 Hz), 7.12 (1H, t, J=7.5 Hz), 7.18 (1H, d, J=8.4 Hz), 7.46 (1H, dd, J=7.8 Hz, 1.5 Hz), 7.53 (1H, m), 7.79 (1H, s); FAB MS m/z 386 [MH+]

### Example 14

¹H NMR (300 MHz, DMSO-d₆) δ 3.86 (3H, s), 3.92 (3H, s), 5.13 (2H, s), 5.99 (2H, s), 6.68-6.93 (5H, m), 7.41 (1H, d, J=8.5 Hz), 7.92 (1H, s); FAB MS m/z 416 [MH+]

### Example 15

¹H NMR (300 MHz, DMSO-d₆) δ 3.83 (s, 3H), 5.13 (s, 2H), 5.98 (s, 2H), 6.81 (dd, 1H, J=8.0 Hz, 1.4 Hz), 6.85 (d, 1H, J=8.0 Hz), 6.90 (d, 1H, J=1.4 Hz), 7.12 (d, 2H, J=9.0 Hz), 7.62 (d, 2H, J=9.0 Hz), 7.81 (s, 1H); EI MS m/z 385 [M+].

### Example 16

¹H NMR (300 MHz, DMSO-d₆) δ 3.82 (s, 3H), 3.84 (s, 3H), 5.13 (s, 2H), 5.98 (s, 2H), 6.81 (dd, 1H, J=8.0 Hz, 1.3 Hz), 6.86 (d, 1H, J=8.0 Hz), 6.91 (d, 1H, J=1.3 Hz), 7.21 (d, 1H, J=8.4 Hz), 7.22 (dd, 1H, J=5.7 Hz, 1.9 Hz), 7.26 (d, 1H, J=2.0 Hz), 7.80 (s, 1H). EI MS m/z 415 [M+].

### Example 17

¹H NMR (300 MHz, DMSO-d₆) δ 1.37 (t, 3H, J=6.9 Hz), 4.10 (q, 2H), 5.14 (s, 2H), 5.99 (s, 2H), 6.82 (dd, 1H, J1=8.0 Hz, 1.4 Hz), 6.86 (d, 1H, J=8.0 Hz), 6.91 (d, 1H, J=1.4 Hz), 6.97 (d, 1H, J=8.4 Hz), 7.14 (dd, 1H, J=8.4 Hz, 1.8 Hz), 7.18 (d, 1H, J=1.8 Hz), 7.76 (s, 1H), 10.08 (s, 1H). EI MS m/z 415 [M+].

### Example 18

¹H NMR (300 MHz, DMSO-d₆) δ 3.85 (s, 3H), 5.15 (s, 2H), 5.99 (s, 2H), 6.82 (dd, 1H, J=8.1 Hz, 1.4 Hz), 6.87 (d, 1H, J=8.1 Hz), 6.92 (d, 1H, J=1.4 Hz), 6.96 (d, 1H, J=8.3 Hz), 7.15 (dd, 1H, J=8.3 Hz, 2.0 Hz), 7.21 (d, 1H, J=2.0 Hz), 7.77 (s, 1H), 10.15 (s, 1H). EI MS m/z 401.

### Example 19

¹H NMR (300 MHz, DMSO-d₆) δ 5.16 (s, 2H), 5.99 (s, 2H), 6.81-6.94 (m, 3H), 7.21-7.30 (m, 2H), 7.52 (d, 1H, J=7.6 Hz), 7.92-7.98 (m, 2H), 8.13 (s, 1H), 12.39 (s, 1H). EI MS m/z 394 [M+].

### Example 20

¹H NMR (300 MHz, DMSO-d₆) δ 3.82 (s, 3H), 3.87 (s, 3H), 5.15 (s, 2H), 6.00 (s, 2H), 6.84 (dd, J=8.2 Hz, 1.0 Hz), 6.88 (d, 1H, J=8.2 Hz), 6.94 (d, 1H, J=1.0 Hz), 7.06-7.09 (m, 1H), 7.25 (d, 1H, J=2.1 Hz), 7.27 (s, 1H), 7.92 (s, 1H). FAB MS m/z 416 [M+1].

### Example 21

¹H NMR (300 MHz, DMSO-d₆) δ 5.15 (s, 2H), 5.99 (s, 2H), 6.83 (dd, 1H, J=8.0 Hz, 1.4 Hz), 6.87 (d, 1H, J=8.0 Hz), 6.92 (d, 1H, J=1.4 Hz), 7.62 (dd, 2H, J=6.6 Hz, 2.2 Hz), 7.68 (dd, 2H, J=6.6 Hz, 2.2 Hz), 7.85 (s, 1H). EI MS m/z 389 [M+].

### Example 22

¹H NMR (300 MHz, DMSO-d₆) δ 5.15 (s, 2H), 6.00 (s, 2H), 6.84 (dd, 1H, J=7.9 Hz, 1.5 Hz), 6.88 (d, 1H, J=7.9 Hz), 6.93 (d, 1H, J=1.5 Hz), 7.57-7.59 (m, 3H), 7.75 (s, 1H), 7.85 (s, 1H). EI MS m/z 389 [M+].

### Example 23

¹H NMR (300 MHz, DMSO-d₆) δ 5.15 (s, 2H), 6.00 (s, 2H), 6.83 (dd, 1H, J=7.9 Hz, 1.2 Hz), 6.88 (d, 1H, J=7.9 Hz), 6.93 (d, 1H, J=1.2 Hz), 7.83 (d, 2H, J=8.4 Hz), 7.91 (s, 1H), 8.01 (d, 2H, J=8.4 Hz). FAB MS m/z 380 [M+1].

### Example 24

¹H NMR (300 MHz, DMSO-d₆) δ 5.16 (s, 2H), 6.00 (s, 2H), 6.83-6.93 (m, 3H), 7.74-7.79 (m, 1H), 7.88-7.99 (m, 3H), 8.15 (s, 1H). EI MS m/z 380 [M+].

### Example 25

¹H NMR (300 MHz, DMSO-d₆) δ 5.13 (s, 2H), 5.99 (s, 2H), 6.82 (dd, 1H, J=8.1 Hz, 1.5 Hz), 6.85-6.88 (m, 3H), 6.91 (s, 1H), 7.04-7.11 (m, 2H), 7.67 (s, 1H), 9.80 (bs, 2H). EI MS m/z 387 [M+].

### Example 26

¹H NMR (300 MHz, DMSO-d₆) δ 3.85 (s, 3H), 5.14 (s, 2H), 5.99 (s, 2H), 6.83 (dd, 1H, J=7.9 Hz, 1.7 Hz), 6.87 (d, 1H, J=7.9 Hz), 6.92 (m, 1H), 7.07 (d, 1H, J=2.2 Hz), 7.10 (d, 1H, J=8.5 Hz), 7.17 (dd, 1H, J=8.5 Hz, 2.2 Hz), 7.71 (s, 1H), 9.59 (s, 1H). EI MS m/z 401 [M+].

### Example 27

¹H NMR (300 MHz, DMSO-d₆) δ 3.94 (s, 3H), 5.15 (s, 2H), 5.99 (s, 2H), 6.83 (d, 1H, J=8.0 Hz), 6.87 (d, 1H, J=8.0 Hz), 6.92 (s, 1H), 7.02 (d, 1H, J=8.8 Hz), 7.86 (s, 1H), 7.93 (dd, 1H, J=8.8 Hz, 2.3 Hz), 8.57 (d, 1H, J=2.3 Hz). FAB MS m/z 387 [M+1].

### Example 28

¹H NMR (300 MHz, DMSO-d₆) δ 5.15 (s, 2H), 5.99 (s, 2H), 6.82-6.92 (m, 3H), 7.19-7.25 (m, 3H), 7.27-7.30 (m, 1H), 7.43-7.49 (m, 2H), 7.86 (s, 1H), 8.06 (dd, 1H, J=8.7 Hz, 2.0 Hz), 8.50 (d, 1H, J=2.0 Hz). EI MS m/z 448 [M+].

### Example 29

¹H NMR (300 MHz, DMSO-d₆) δ 2.56 (s, 3H), 5.14 (s, 2H), 6.00 (s, 2H), 6.81-6.94 (m, 3H), 7.33-7.38 (m, 1H), 7.45-7.51 (m, 3H), 7.99 (s, 1H). EI MS m/z 401 [M+].

### Example 30

¹H NMR (300 MHz, DMSO-d₆) δ 5.15 (s, 2H), 5.99 (s, 2H), 6.84 (dd, 1H, J=8.0 Hz, 1.1 Hz), 6.87 (d, 1H, J=8.9 Hz), 6.93 (d, 1H, J=1.1 Hz), 7.71 (d, 2H, J=8.4 Hz), 7.88 (s, 1H), 8.05 (d, 2H, J=8.4 Hz). EI MS m/z 399 [M+].

### Example 31

¹H NMR (300 MHz, DMSO-d₆) δ 5.15 (s, 2H), 5.99 (s, 2H), 6.84 (dd, 1H, J=8.0 Hz, 1.4 Hz), 6.87 (d, 1H, J=8.0 Hz), 6.93 (d, 1H, J=1.4 Hz), 7.57 (dd, 2H, J=4.6 Hz, 1.6 Hz), 7.81 (s, 1H), 8.75 (dd, J=4.6 Hz, 1.6 Hz). EI MS m/z 356 [M+].

### Example 32

¹H NMR (300 MHz, DMSO-d₆) δ 5.15 (s, 2H), 6.00 (s, 2H), 6.84-6.94 (m, 3H), 7.42-7.48 (m, 1H), 7.59 (d, 2H, J=4.2 Hz), 7.84 (d, 1H, J=7.8 Hz), 7.90 (s, 1H). EI MS m/z 433 [M+].

### Example 33

¹H NMR (300 MHz, DMSO-d₆) δ 5.15 (s, 2H), 6.00 (s, 2H), 6.81-6.94 (m, 3H), 7.59-7.63 (m, 2H), 7.71-7.78 (m, 1H), 8.02 (d, 1H, J=7.8 Hz), 8.46 (s, 1H). EI MS m/z 399 [M+].

### Example 34

¹H NMR (300 MHz, DMSO-d₆) δ 4.03 (s, 3H), 5.18 (s, 2H), 6.00 (s, 2H), 6.85 (dd, 1H, J=7.9 Hz, 1.5 Hz), 6.88 (d, 1H, J=7.9 Hz), 6.94 (d, 1H, J=1.5 Hz), 7.30-7.43 (m, 2H), 7.69 (d, 1H, J=7.9 Hz), 7.80 (d, 1H, J=7.9 Hz), 7.88 (s, 1H). EI MS m/z 409 [M+].

### Example 35

¹H NMR (300 MHz, DMSO-d₆) δ 5.16 (s, 2H), 6.00 (s, 2H), 6.84 (dd, 1H, J=7.9 Hz, 1.2 Hz), 6.88 (d, 1H, J=7.9 Hz), 6.93 (d, 1H, J=1.2 Hz), 7.97 (s, 1H), 8.06 (d, 1H, J=8.1 Hz), 8.27 (dd, 1H, J=8.1 Hz, 1.8 Hz), 9.01 (d, 1H, J=1.8 Hz). EI MS m/z 424 [M+].

### Example 36

¹H NMR (300 MHz, DMSO-d₆) δ 5.13 (s, 2H), 5.99 (s, 2H), 6.82 (dd, 1H, J=8.0 Hz, 1.1 Hz), 6.86 (d, 1H, J=8.0 Hz), 6.92 (d, 1H, J=1.1 Hz), 6.98 (dd, 1H, J=8.6 Hz, 1.8 Hz), 7.20 (d, 1H, J=1.8 Hz), 7.72 (s, 1H), 7.93 (d, 1H, J=8.6 Hz). EI MS m/z 416 [M+].

### Example 37

¹H NMR (300 MHz, DMSO-d₆) δ 5.16 (s, 2H), 6.00 (s, 2H), 6.79-6.93 (m, 3H), 7.68 (t, 1H, J=7.7 Hz), 7.89 (d, 1H, J=8.1 Hz), 7.93 (s, 1H), 8.05 (d, 1H, J=7.5 Hz), 8.18 (s, 1H). EI MS 399 [M+].

### Example 38

¹H NMR (300 MHz, DMSO-d₆) δ 5.15 (s, 2H), 6.00 (s, 2H), 6.83-6.93 (m, 3H), 7.58 (t, 1H, J=5.7 Hz), 7.72 (s, 1H), 8.61 (d, 1H, J=4.8 Hz), 8.77 (d, 1H, J=2.1 Hz). EI MS m/z 374 [M+].

### Example 39

¹H NMR (300 MHz, DMSO-d₆) δ 5.14 (s, 2H), 5.99 (s, 2H), 6.16 (s, 2H), 6.81-6.92 (m, 3H), 7.10-7.24 (m, 3H), 7.78 (s, 1H). EI MS m/z 399 [M+].

### Example 40

¹H NMR (300 MHz, DMSO-d₆) δ 5.16 (s, 2H), 6.00 (s, 2H), 6.83-6.94 (m, 3H), 8.12 (s, 1H), 9.70 (s, 1H). EI MS m/z 363 [M+].

### Example 41

¹H NMR (300 MHz, DMSO-d₆) δ 5.14 (s, 2H), 5.99 (s, 2H), 6.82 (dd, 1H, J=8.0 Hz, 1.4 Hz), 6.87 (d, 1H, J=8.0 Hz), 6.92 (d, 1H, J=1.4 Hz), 8.09 (s, 1H), 8.11 (d, 1H, J=3.0 Hz), 8.24 (d, 1H, J=3.0 Hz). EI MS m/z 362 [M+].

### Example 42

¹H NMR (300 MHz, DMSO-d₆) δ 5.17 (s, 2H), 6.00 (s, 2H), 6.84 (dd, 1H, J=7.9 Hz, 1.0 Hz), 6.88 (d, 1H, J=7.9 Hz), 6.94 (d, 1H, J=1.0 Hz), 7.86 (d, 2H, J=8.9 Hz), 7.91 (s, 1H), 8.07 (d, 2H, J=8.9 Hz), 8.31 (s, 1H), 9.43 (s, 1H). EI MS m/z 422 [M+].

### Example 43

¹H NMR (300 MHz, DMSO-d₆) δ 5.12 (s, 2H), 5.99 (s, 2H), 6.82 (dd, 1H, J=7.9 Hz, 1.5 Hz), 6.86 (d, 1H, J=7.9 Hz), 6.91 (d, 1H, J=1.5 Hz), 7.39 (s, 1H), 7.51 (s, 1H), 7.53 (s, 1H), 12.97 (s, 1H). EI MS m/z 345 [M+].

### Example 44

¹H NMR (300 MHz, DMSO-d₆) δ 3.18 (t, 4H, J=4.4 Hz), 3.77 (t, 4H, J=4.2 Hz), 5.15 (s, 2H), 6.00 (s, 2H), 6.87-6.95 (m, 3H), 7.33 (d, 1H, J=7.3 Hz), 7.72 (s, 1H), 8.26 (d, 1H, J=2.7 Hz), 8.30 (dd, 1H, J=7.3 Hz, 2.7 Hz). EI MS m/z 485 [M+].

### Example 45

¹H NMR (300 MHz, DMSO-d₆) δ 5.14 (s, 2H), 5.51 (s, 2H), 5.99 (s, 2H), 6.83 (dd, 1H, J=8.0 Hz, 1.3 Hz), 6.86 (d, 1H, J=8.0 Hz), 6.92 (d, 1H, J=1.3 Hz), 7.42 (d, 2H, J=8.3 Hz), 7.64 (d, 2H, J=8.3 Hz), 7.82 (s, 1H), 8.01 (s, 1H), 8.69 (s, 1H). EI MS m/z 436 [M+].

### Example 46

¹H NMR (300 MHz, DMSO-d₆) δ 4.99 (s, 2H), 5.14 (s, 2H), 5.41 (s, 2H), 5.99 (s, 2H), 6.83 (d, 1H, J=8.0 Hz), 6.87 (d, 1H, J=8.0 Hz), 6.92 (s, 1H), 7.16 (d, 1H, J=9.0 Hz), 7.20 (d, 1H, J=9.0 Hz), 7.82 (s, 1H). EI MS m/z 431 [M+].

### Example 47

¹H NMR (300 MHz, DMSO-d₆) δ 3.50 (t, 4H, J=4.8 Hz), 3.72 (t, 4H, J=4.8 Hz), 5.15 (s, 2H), 6.00 (s, 2H), 6.81 (dd, 1H, J=6.6 Hz, 1.2 Hz), 6.84 (d, 1H, J=1.2 Hz), 6.87 (d, 1H, J=6.6 Hz), 6.92 (d, 1H, J=1.3 Hz), 7.04 (s, 1H), 7.72 (s, 1H), 8.28 (d, 1H, J=5.1 Hz). EI MS m/z 441 [M+].

### Example 48

¹H NMR (300 MHz, DMSO-d₆) δ 5.16 (s, 2H), 6.00 (s, 2H), 6.85 (dd, 1H, J=7.9 Hz, 1.4 Hz), 6.88 (d, 1H, J=7.9 Hz), 6.94 (d, 1H, J=1.4 Hz), 7.39-7.43 (m, 1H), 7.77 (d, 2H, J=8.6 Hz), 7.90 (s, 1H), 7.94 (dd, 1H, J=7.7 Hz, 1.8 Hz), 8.07 (d, 1H, J=8.1 Hz), 8.28 (d, 2H, J=8.6 Hz), 8.70-8.72 (m, 1H). EI MS m/z 432 [M+].

### Example 49

¹H NMR (300 MHz, DMSO-d₆) δ 3.07 (s, 3H), 5.15 (s, 2H), 5.99 (s, 2H), 6.83 (dd, 1H, J=8.0 Hz, 1.1 Hz), 6.87 (d, 1H, J=8.0 Hz), 6.93 (d, 1H, J=1.1 Hz), 7.32 (d, 1H, J=7.8 Hz), 7.40 (d, 1H, J=8.1 Hz), 7.45 (s, 1H), 7.51 (t, 1H, J=8.0 Hz), 7.80 (s, 1H), 10.35 (bs, 1H). EI MS m/z 448 [M+].

### Example 50

¹H NMR (300 MHz, DMSO-d₆) 6 (ppm) 3.18 (t, 4H, J=4.4 Hz), 3.71 (t, 4H, J=4.4 Hz), 5.15 (s, 2H), 6.00 (s, 2H), 6.85-6.93 (m, 3H), 7.42 (d, 1H, J=9 Hz), 7.77 (dd, 1H, J=9 Hz, 2.3 Hz), 7.85 (s, 1H), 8.17 (d, 1H, J=2.3 Hz). EI MS m/z 485 [M+].

### Example 51

¹H NMR (300 MHz, DMSO-d₆) 6 (ppm) 1.60 (s, 6H), 5.13 (s, 2H), 6.75-6.84 (m, 3H), 7.81-7.86 (m, 1H), 8.01 (s, 1H), 8.03-8.06 (m, 1H), 8.32 (ddd, 1H, J=8.2 Hz, 2.3 Hz, 0.9 Hz), 8.50-8.51 (m, 1H). EI MS m/z 428 [M+].

### Example 52

¹H NMR (300 MHz, DMSO-d₆) δ (ppm) 1.60 (s, 6H), 5.11 (s, 2H), 6.74-6.84 (m, 3H), 6.91 (ddd, 1H, J=8.1 Hz, 2.38 Hz, 0.82 Hz), 7.00-7.02 (m, 1H), 7.07-7.11 (m, 1H), 7.32-7.37 (m, 1H), 7.74 (s, 1H). EI MS m/z 399 [M+].

### Example 53

¹H NMR (300 MHz, DMSO-d₆) δ (ppm) 5.23 (s, 2H), 6.92 (ddd, 1H, J=8.2 Hz, 2.4 Hz, 0.8 Hz), 7.01-7.03 (m, 1H), 7.08-7.09 (m, 1H), 7.13 (dd, 1H, J=8.4 Hz, 1.7 Hz), 7.33-7.41 (m, 3H), 7.75 (s, 1H), 9.90 (s 1H). EI MS m/z 407 [M+].

### Example 54

¹H NMR (300 MHz, DMSO-d₆) δ (ppm) 4.20 (s, 4H), 5.12 (s, 2H), 6.81-6.85 (m, 3H), 7.81-7.86 (m, 1H), 8.01-8.06 (m, 2H), 8.32 (d, 1H, J=8.1 Hz), 8.50 (s 1H). EI MS m/z 414 [M+].

### Example 55

¹H NMR (300 MHz, DMSO-d₆) δ (ppm) 4.80 (s, 2H), 6.07 (s, 2H), 6.89-7.11 (m, 5H), 7.32-7.38 (m, 1H), 7.85 (s, 1H), 9.87 (s, 1H). EI MS m/z 418 [M+].

### Example 56

¹H NMR (300 MHz, DMSO-d₆) δ (ppm) 4.60 (s, 2H), 5.90-5.98 (m, 4H), 6.52 (d, 1H, J=7.9 Hz), 6.84 (s, 1H), 7.42 (d, 1H, J=7.8 Hz), 7.45-7.54 (m, 1H), 7.83 (s, 1H), 8.07 (bs, 1H), 8.18-8.21 (m, 1H). EI MS m/z 367 [M+].

### Example 57

¹H NMR (300 MHz, DMSO-d₆) δ (ppm) 4.74 (s, 2H), 5.99 (s, 2H), 6.81 (dd, 1H, J=8.1 Hz, 1.7 Hz), 6.86 (s, 1H), 6.88 (d, 1H, J=1.8 Hz), 7.80-7.85 (m, 1H), 8.02-8.04 (m, 1H), 8.11 (s, 1H), 8.30 (ddd, 1H, J=8.3 Hz, 2.3 Hz, 0.9 Hz), 8.47-8.48 (m, 1H). EI MS m/z 384 [M+].

### Example 58

¹H NMR (300 MHz, DMSO-d₆) δ (ppm) 4.73 (s, 2H), 6.00 (s, 2H), 6.80 (dd, 1H, J=8.1 Hz, 1.6 Hz), 6.86-6.92 (m, 3H), 7.00-7.05 (m, 2H), 7.30-7.35 (m, 1H), 7.85 (s, 1H). ES+MS m/z 356 [MH+].

### Example 59

¹H NMR (300 MHz, DMSO-d₆) δ (ppm) 3.01 (s, 6H), 4.70 (s, 2H), 5.98 (s, 2H), 6.78-6.87 (m, 5H), 7.45 (d, 2H, J=8.9 Hz), 7.81 (s, 1H). ES+MS m/z 383 [MH+].

### Example 60

¹H NMR (300 MHz, DMSO-d₆) δ (ppm) 3.70 (s, 2H), 4.57 (s, 2H), 5.97 (s, 2H), 6.76-6.86 (m, 4H), 7.00 (s, 1H), 7.06 (d, 1H, J=7.8), 7.24-7.29 (m, 1H), 7.37 (s, 1H), 9.66 (s, 1H). EI MS m/z 337 [M+].

### Example 61

¹H NMR (300 MHz, DMSO-d₆) δ (ppm) 4.91 (s, 2H), 5.98 (s, 2H), 6.78-6.93 (m, 4H), 7.67-7.72 (m, 1H), 8.12 (d, 1H, J=7.8 Hz), 8.19 (dd, 1H, J1=9.7 Hz, 1.53 Hz), 8.49 (s, 1H), 12.74 (bs, 1H). EI MS m/z 383 [M+].

### Example 62

### Sythesis of 3-((2,3-dihydrobenzo[b][1,4]dioxin-6-yl)methyl)-5-(3-nitrobenzylidene)imidazolidine-2,4-dione (compound of Example 54)

### Step 1. Preparation of 3-((2,3-dihydrobenzo[b][1,4]dioxin-6-yl)methyl)-2-imidazolidin-4-one

A suspension of (2,3-Dihydro-benzo[1,4]dioxin-6-yl)-methylamine (16.5 mmol, 2.73 g) in 1M NaOH is refluxed for 10 min and cooled to room temperature. Subsequently bis(carboxymethyl) trithiocarbonate (3.74 g, 16.5 mmol) is added and the reaction mixture refluxed overnight to give the precipitate, which is filtered off and is washed with water (3×20 mL). The remaining residue is suspended in a mixture of methanol and isopropanol (1:1, 25 mL) and refluxed for 10 minutes. Cooled reaction mixture is filtered and the solid residue dried at lower temperature and recrystallyzed from methanol to give 3.29 g of 3-((2,3-dihydrobenzo[b][1,4]dioxin-6-yl)methyl)-imidazolidin-2,4-dione.

### Step 2. Coupling of aldehydes or ketones to 3-((2,3-dihydrobenzo[b][1,4]dioxin-6-yl)methyl)-imidazolidin-2,4-dione

A solution of 74.5 mg (=0.3 mmol) 3-(benzo[d][1,3]dioxol-5-ylmethyl)-2-thioxothiazolidin-4-one, corresponding aldehyde R1HCO (0.3 mmol) and sodium acetate (127.4 mg, 1.50 mmol) in acetic acid (3 mL) is heated in the microwave reactor for 2 h at around 130° C. The reaction mixture is cooled to room temperature and the obtained precipitate filtered off and washed with EtOH (2×5 mL) and MeOH (2×5 mL) and dried at elevated temperature.

### Example 63

### 3-(benzo[d][1,3]dioxol-5-ylmethyl)-5-(3-nitrobenzylidene)imidazo-lidine-2,4-dione

### Step 1. Synthesis of 5-(3-nitrobenzylidene)imidazolidine-2,4-dione

Coupling of aldehydes to imidazolidin-2,4-dione is performed in a similar manner as described in example 62.

### Step 2. Synthesis of 3-(benzo[d][1,3]dioxol-5-ylmethyl)-5-(3-nitrobenzylidene)imidazolidine-2,4-dione

DBU (3.82 mmol, 0.961 g) is added into solution of 5-(3-nitrobenzylidene)imidazolidine-2,4-dione (3.19 mmol, 0.743 g) at 0° C. and the solution left to warm to room temperature. 5-(Chloromethyl)benzo[d][1,3]dioxole (6.37 mmol, 2.174 g, 50% soln in CH₂Cl₂) is added and the reaction mixture stirred for 3 hours. Subsequently 0.5 M HCL solution is added and extracted with ethylacetate (2×50 mL) dried over MgSO₄ and the solvent removed under reduced pressure. The obtained product is recrystallized from diisopropylether to afford 0.13 (11%) of 3-(benzo[d][1,3]dioxol-5-ylmethyl)-5-(3-nitrobenzylidene)imidazo-lidine-2,4-dione.

### Example 64

### Synthesis of 3-(benzo[d][1,3]dioxol-5-ylmethyl)thiazolidine-2,4-dione

A suspension of thiazolidine-2,4-dione (10.0 mmol, 1.17 g), 5-(chloromethyl)benzo[d][1,3]dioxole (10.0 mmol, 50% soln in CH₂Cl₂) and potassium fluoride (10.0 mmol, 0.581 g) is stirred at room temperature overnight. The solvent is removed under reduced pressure and the residue dissolved in H₂O (50 mL) and extracted into dichloromethane (3×20 mL). Combined organic fractions were dried over Na₂SO₄ and the solvent removed under reduced pressure. The obtained oily product is washed with diethyl ether to give 1.590 g (64%) of 3-(benzo[d][1,3]dioxol-5-ylmethyl)thiazolidine-2,4-dione as a white precipitate.

### General Procedure D: Sythesis of 1-(benzo[d][1,3]-dioxol-5-ylmethyl)-3-substituted-pyrrolidine-2,5-dione

### Step 1. Synthesis of 1-(benzo[d][1,3]dioxol-5-ylmethyl)-1H-pyrrole-2,5-dione

Diethylazodicarboxylate (20.0 mmol, 3.15 mL) is added into a solution of maleimide (20.0 mmol, 1.94 g), piperonyl alcohol (20.0 mmol, 3.043 g) and triphenylphosphine (20.0 mmol, 5.246 g) in THF (100 mL) and stirred at room temperature for 1 hour. The solvent is removed under reduced pressure and the residue purified by column chromatography (silicagel; ethylacetate/hexane=½) to yield 2.26 g (49%) of 1-(benzo[d][1,3]dioxol-5-ylmethyl)-1H-pyrrole-2,5-dione.

### Step 2. Coupling of aldehydes to 1-(benzo[d][1,3]dioxol-5-ylmethyl)-1H-pyrrole-2,5-dione

A solution of 1-(benzo[d][1,3]dioxol-5-ylmethyl)-1H-pyrrole-2,5-dione (0.87 mmol, 0.20 g) and triphenylphosphine (0.87 mmol, 0.227 g) in acetic acid is heated for 1 hour at 110° C. and the solvent removed under reduced pressure. The remaining oily residue is suspended in toluene (3 mL), a corresponding aldehyde (0.87 mmol) is added and heated the reaction mixture for 30 minutes at 100° C. The obtained precipitate is filtered off, washed with THF and dried at elevated temperature.

### Example 65

### Synthesis of 3-(benzo[d][1,3]dioxol-5-ylmethyl)-2-thioxoimidazolidin-4-one

A solution of glycine ethyl ester hydrochloride (2.0 mmol, 0.297 g), 5-(isothiocyanatomethyl)benzo[d][1,3]dioxole (2.00 mmol, 0.386 g) and sodium hydrogencarbonate (3.0 mmol, 0.252 g) in DMF (10 mL) is stirred at room temperature for 1 hour and then at 65° C. overnight. The solvent is removed under reduced pressure and the oily residue purified by column chromatography (silicagel; ethylacetate/hexane=½) to yield 0.339 g (68%) of 3-(benzo[d][1,3]dioxol-5-ylmethyl)-2-thioxoimidazolidin-4-one.

### Example 66

### Enzyme Assays and IC₅₀-determination

### Method A: MurD Enzyme Assay

The compounds are tested for their ability to inhibit the formation of UDP-MurNAc-L-Ala-D-[¹⁴C]Glu in mixtures (50 μl) containing 0.1 M Tris-HCl, pH 8.6, 5 mM MgCl₂, 5 mM ATP, 25 μM UDP-MurNAc-L-Ala, 25 μM D-[¹⁴C]Glu, inhibitor (100 μM), DMSO (5%, v/v), and enzyme. After 30 min at 37° C., the reaction is stopped by addition of acetic acid (10 μl). After lyophilization, D-[¹⁴C]Glu and of UDP-MurNAc-L-Ala-D-[¹⁴C]Glu are separated by reverse-phase HPLC and quantitated by on-line scintillation counting. The residual activity is determined with respect to an assay with 5% DMSO and without inhibitor (taken as 100%). Values presented are means of duplicates.

IC₅₀ values are determined with several concentrations of the inhibitor using the Hill equation:

log[vᵢ/(vₒ−vᵢ)]=−n log[l]+logK′

where vₒ is the rate without inhibitor, vᵢ the rate in the presence of inhibitor and n the Hill number.

### Method B: MurE Enzyme Assay

The compounds are tested for their ability to inhibit the formation of UDP-MurNAc-L-Ala-γ-D-Glu-L-[¹⁴C]Lys in mixtures (50 μl) containing 0.1 M Tris-HCl, pH 8.6, 15 mM MgCl₂, 5 mM ATP, 100 μM UDP-MurNAc-L-Ala-γ-D-Glu, 200 μM L-[¹⁴C]Lys (50,000 cpm), inhibitor (100 μM), DMSO (5%, v/v), and enzyme from S. aureus. After 30 min at 37° C., the reaction is stopped by addition of acetic acid (10 μl). After lyophilization, L-[¹⁴C]Lys and of UDP-MurNAc-L-Ala-Y-D-Glu-L-[¹⁴C]Lys are separated by reverse-phase HPLC on Nucleosil 100C18 5U (150×4.6 mm) in 50 mM ammonium formate, pH 4.7, at 0.6 ml/min, and quantitated by on-line scintillation counting using the Quicksafe Flow 2 scintillating fluid. The residual activity is determined with respect to an assay with 5% DMSO and without inhibitor (taken as 100%). Values presented are means of duplicates.

IC₅₀ values were determined with several concentrations of the inhibitor using the Hill equation:

log[vᵢ/(vₒ−vᵢ)]=−n log[l]+logK′

where vₒ is the rate without inhibitor, vᵢ the rate in the presence of inhibitor and n the Hill number.

TABLE 2
Activities of representative compounds tested in
an E. coli MurD assay and S. aureus MurE assay.

| Compound | E. coli MurD | | S. aureus MurE | |
|---|---|---|---|---|
| of Example | RA at 100 μM | | RA at 100 μM | |
| No. | inhibitor conc. | IC₅₀ (μM) | inhibitor conc. | IC₅₀ (μM) |
| 1 | 77% | >100 | 82% | >100 |
| 2 | 22% | 111 | 7% | 4 |
| 3 | 7% | 24 | 4% | 5 |
| 4 | 26% | 15 | 4% | 4 |
| 5 | 71% | >100 | 10% | 14 |
| 6 | 99% | >100 | 5% | 37 |
| 7 | 98% | >100 | 19% | 7 |
| 8 | 100% | >100 | 23% | 1.5 |
| 9 | 98% | >100 | 17% | 51 |
| 10 | 97% | >100 | 31% | 13 |
| 11 | 95% | >100 | 25% | 4.5 |
| 12 | 78% | >100 | 59% | >100 |
| 13 | 75% | >100 | 90% | >100 |
| 14 | 72% | >100 | 67% | >100 |

1. A compound of formula 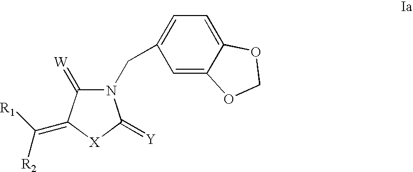
wherein
X denotes S,
Y denotes S,
W denotes O,
R1 is hydrogen and R2 is a group of formula 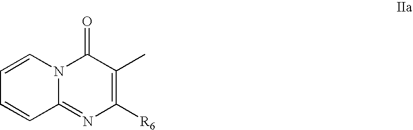
wherein R6 denotes amino, alkylamino, dialkylamino, (hydroxyalkyl)amino, di(hydroxyalkyl)amino, (heterocyclylalkyl)amino, (hydroxyalkoxyalkyl)amino or di(hydroxyalkoxyalkyl)amino,
and pharmaceutically acceptable salts of a compound of formula Ia.

2. The compound according to claim 1 wherein R6 is methylamino, ((tetrahydrofuran-2-yl)-methyl)amino, (hydroxy)(lower alkyl))amino or (2-hydroxyethoxy)ethyl)amino.

3. The compound according to claim 1 wherein R6 is ((tetrahydrofuran-2-yl)-methyl)amino.

4. A method of treating or preventing a disease or disorder comprising administering a compound to a subject in need thereof, wherein the compound comprises 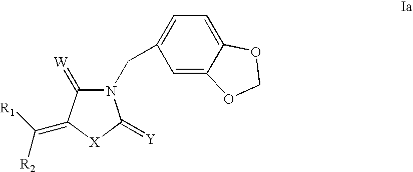
wherein
X denotes S,
Y denotes S,
W denotes O,
R1 is hydrogen and R2 is a group of formula 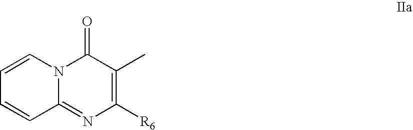
wherein R6 denotes amino alkylamino, dialkylamino, (hydroxyalkyl)amino, di(hydroxyalkyl)amino, (heterocvclylalkyl)amino, (hydroxyalkoxyalkyl)amino or di(hydroxyalkoxyalkyl)amino,
and pharmaceutically acceptable salts of a compound of formula Ia.

5. The method according to claim 4 wherein the compound of formula Ia or a pharmaceutically acceptable salt thereof is administered as an antibiotic.

6. The method according to claim 4 wherein the compound of formula Ia or a Pharmaceutically acceptable salt thereof is administered for the treatment of a bacterial disease.

7. The method according to claim 6 wherein the bacterial disease is caused by a bacterium strain exhibiting MurD and/or MurD ligase activity.

8. A process for the preparation of a compound of formula Ia comprising 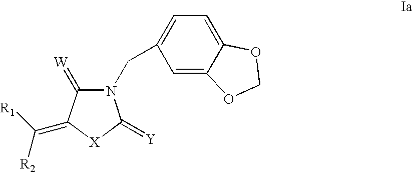
wherein
X denotes S,
Y denotes S,
W denotes O,
R1 is hydrogen and R2 is a group of formula 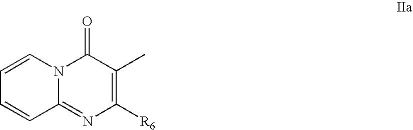
wherein R6 denotes amino alkylamino, dialkylamino, (hydroxyalkyl)amino, di(hydroxyalkyl)amino, (heterocyclylalkyl)amino, (hydroxyalkoxyalkyl)amino or di(hydroxyalkoxyalkyl)amino, the method comprising the steps of
a) reacting a compound of formula 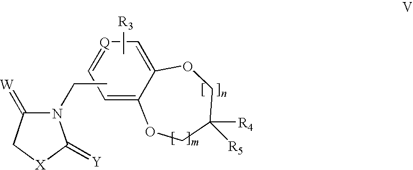
wherein
X denotes S,
Y denotes S,
W denotes O,
Q denotes CH,
n is 0,
m is 0,
R3 denotes hydrogen, and
R4 and R5 denote independently from each other hydrogen,
with an aldehyde or ketone of formula 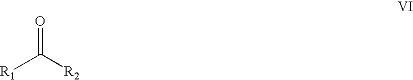
wherein R1 and R2 are defined as in formula Ia to obtain a compound of formula Ia, and optionally
c) isolating a compound of formula Ia.

9. A process for the preparation of a compound of formula Ia comprising 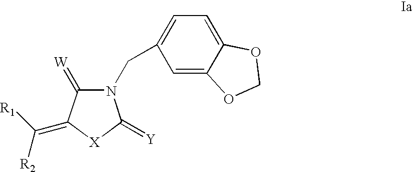
wherein
X denotes S,
Y denotes S,
W denotes O,
R1 is hydrogen and R2 is a group of formula 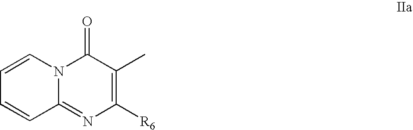
wherein R6 denotes amino alkylamino, dialkylamino, (hydroxyalkyl)amino, di(hydroxyalkyl)amino, (heterocyclylalkyl)amino, (hydroxyalkoxyalkyl)amino or di(hydroxyalkoxyalkyl)amino, the method comprising the steps of
t) reacting a compound of formula 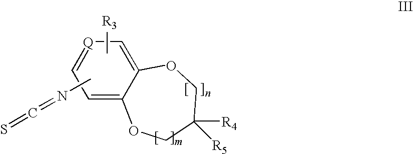
wherein
Q denotes CH,
n is 0,
m is 0,
R3 denotes hydrogen, and
R4 and R5 denote independently from each other hydrogen,
with a compound of formula 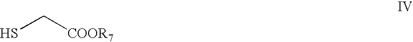
wherein R7 denotes alkyl,
to obtain a compound of formula 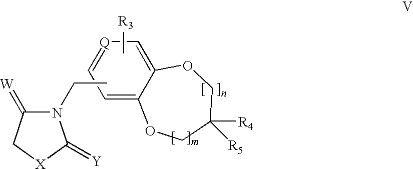
wherein
X denotes S,
Y denotes S,
W denotes O,
Q denotes CH,
n is 0,
m is 0,
R3 denotes hydrogen, and
R4 and R5 denote independently from each other hydrogen,
b) reacting a compound of formula V as defined in step t) with an aldehyde or ketone of formula 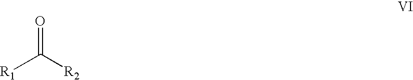
wherein R1 and R2 are defined as in formula Ia, to obtain a compound of formula Ia, and optionally
c) isolating a compound of formula Ia.

10. A pharmaceutical composition comprising a compound of formula Ia or a pharmaceutically acceptable salt thereof and one or more pharmaceutically acceptable excipient(s), wherein the compound of formula 1a comprises 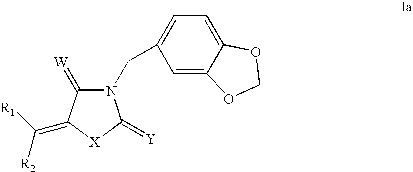
wherein
X denotes S,
Y denotes S,
W denotes O,
R1 is hydrogen and R2 is a group of formula 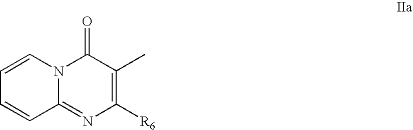
wherein R6 denotes amino alkylamino, dialkylamino, (hydroxyalkyl)amino, di(hydroxyalkyl)amino, (heterocyclylalkyl)amino, (hydroxyalkoxyalkyl)amino or di(hydroxyalkoxyalkyl)amino.
